(19) United States
(12) Patent Application Publication
(10) Pub. No.: US 2025/0285767 A1
(43) Pub. Date: Sep. 11, 2025

(54) ASSESSING RISK FOR MULTIPLE MYELOMA PRECURSOR DISEASE PROGRESSION

(71) Applicants: The Broad Institute, Inc., Cambridge, MA (US); The General Hospital Corporation, Boston, MA (US); Dana-Farber Cancer Institute, Inc., Boston, MA (US)

(72) Inventors: Lorenzo Trippa, Boston, MA (US); Gad Getz, Boston, MA (US); Irene M. Ghobrial, Boston, MA (US); Anna Cowan, Boston, MA (US); Federico Ferrari, Boston, MA (US); Samuel Freeman, Cambridge, MA (US)

(73) Assignees: The Broad Institute, Inc., Cambridge, MA (US); The General Hospital Corporation, Boston, MA (US); Dana-Farber Cancer Institute, Inc., Boston, MA (US)

(21) Appl. No.: 19/219,826

(22) Filed: May 27, 2025

Related U.S. Application Data
(63) Continuation of application No. PCT/US2023/081160, filed on Nov. 27, 2023.
(60) Provisional application No. 63/385,194, filed on Nov. 28, 2022.

(51) Int. Cl. G16H 50/30; G06N 20/00; G16H 10/60; G16H 40/67; G16H 50/70

(52) U.S. Cl. CPC G16H 50/30; G06N 20/00; G16H 10/60; G16H 40/67; G16H 50/70

(57) ABSTRACT

Techniques for estimating a risk that a condition of a patient with a multiple myeloma (MM) precursor disease such as MGUS or SMM will progress into MM.

### CROSS REFERENCE TO RELATED APPLICATIONS

This application is a continuation under 35 U.S.C. § 111 (a) of PCT International Patent Application No. PCT/US2023/081160, filed Nov. 27, 2023, designating the United States and published in English, which claims priority to and the benefit of U.S. Provisional Application No. 63/385,194, filed Nov. 28, 2022, the entire contents of each of which are incorporated by reference herein.

### BACKGROUND OF THE DISCLOSURE

Multiple myeloma (MM) is a cancer that develops in human plasma cells. Plasma cells form a part of the immune system, in particular by making antibodies called immunoglobulins that bind to antigens. In multiple myeloma, the plasma cells may produce an abnormal protein known as monoclonal immunoglobulin, also called “M-spike.”

MM is typically preceded by an MM precursor disease. A patient at risk for MM first develops a medical condition known as monoclonal gammopathy of undetermined significance (MGUS). Following MGUS, the patient may develop smoldering multiple myeloma (SMM).

### SUMMARY OF THE DISCLOSURE

As described below, the present disclosure features techniques for estimating a risk that a condition of a patient with a multiple myeloma (MM) precursor disease such as MGUS or SMM will progress into MM, such as a risk that the patient's condition will progress into MM within a timeframe or across multiple timeframes (e.g., successive time frames).

In one aspect, the disclosure features a method for determining the risk that a patient with a multiple myeloma (MM) precursor disease will progress to MM. The method involves actions a)-c). Action a) involves analyzing the age of the patient and a plurality of values using at least one trained model trained to evaluate risk of an MM precursor disease progressing into MM. The plurality of values contains a plurality of numeric values each being for a corresponding time-varying marker and at least one trajectory value describing a change over time of a time-varying marker. Action b) involves generating, as a result of the analyzing using the at least one trained model, a numeric value indicating the risk that the MM precursor disease of the patient will progress into MM. Action c) involves outputting the value indicating the risk for the patient.

In another aspect, the disclosure features a method involving assessing, with at least one processor and for a patient with a multiple myeloma (MM) precursor disease, a risk that the MM precursor disease of the patient will progress into MM. The assessing involves analyzing, for the patient, an age of the patient together with a plurality of values each representing a level, detected at a time and for the patient, of a time-varying marker of a plurality of time-varying markers. The analyzing involving analyzing the age of the patient and the plurality of values using at least one trained model trained to evaluate risk of MM precursor disease progressing into MM. The plurality of values contains a first plurality of numeric values each being for a corresponding time-varying marker of a plurality of first time-varying markers, and at least one trajectory value each describing a change over time of a corresponding time-varying marker of at least one second time-varying marker. The method further involves, generating, as a result of the analyzing using the at least one trained model, a value indicating the risk that the MM precursor disease of the patient will progress into MM. The method involves outputting the value indicating the risk for the patient.

In another aspect, the disclosure features a computer-implemented method for assessing risk for multiple myeloma precursor disease progression in a subject. The computer-implemented method involves receiving, by at least one server from a computing device via a network, a multiple myeloma precursor disease progression request containing at least one variable representing at least one marker measurement associated with a subject. The method further involves determining, by the at least one server, a Precursor Asymptomatic Neoplasms by Group Effort Analysis (PANGEA) machine learning model from a set of PANGEA machine learning models based at least in part on the at least one variable provided in the multiple myeloma precursor disease progression request. Each PANGEA machine learning model of the set of PANGEA machine learning models contains trained PANGEA parameters trained for a particular combination of variables based at least in part on training data. The training data contains historical marker measurements for the particular combination of variables paired with known trajectories of the particular combination of variables. The method further involves utilizing, by the at least one server, the PANGEA machine learning model to ingest the at least one variable and produce a predicted risk throughout a set of prediction periods representing a future risk of progression for the subject based at least in part on the trained PANGEA parameters. The method also involves transmitting, by the at least one server, a multiple myeloma precursor disease progression response containing the predicted risk throughout the prediction period, the multiple myeloma precursor disease progression response being configured to cause the computing device to render a graphical plot depicting the future risk of progression based on the at least one continuous variable.

In another aspect, the disclosure features at least one computer-readable storage medium having encoded thereon executable instructions that, when executed by at least one processor, cause the at least one processor to carry out the method of any one or any combination of any aspect provided herein, or embodiments thereof.

In another aspect, the disclosure features an apparatus containing at least one processor and at least one computer-readable storage medium having encoded thereon executable instructions that, when executed by the at least one processor, cause the at least one processor to carry out the method of any one or any combination of any aspect provided herein, or embodiments thereof.

In any aspect provided herein, or embodiments thereof, the time-varying markers are clinical variables selected from creatinine, age, hemoglobin, M-spike, serum free light chain (FLC) ratio, bone marrow plasma cell percent (BMPC %), total protein, IgA, IgM, IgG, kappa free light chain (FLC), lambda FLC, calcium, albumin, hemoglobin, LDH, beta-2 microglobulin, and weight.

In any aspect provided herein, or embodiments thereof, the MM precursor disease is monoclonal gammopathy of undetermined significance (MGUS) or smoldering multiple myeloma (SMM).

In any aspect provided herein, or embodiments thereof, the risk that the MM precursor disease of the patient will progress into MM involves assessing the risk that the MM precursor disease of the patient will, within a timeframe, progress into MM.

In any aspect provided herein, or embodiments thereof, assessing the risk that the MM precursor disease of the patient will, within the timeframe, progress into MM, involves assessing a risk for each of a plurality of timeframes that the MM precursor disease will, within the corresponding timeframe, progress into MM.

In any aspect provided herein, or embodiments thereof, generating the value indicating the risk involves generating a numeric value indicating the risk, and outputting the value indicating the risk involves outputting the numeric value.

In any aspect provided herein, or embodiments thereof, assessing the risk further involves determining whether an input value resulting from a bone marrow biopsy has been received. Further, analyzing using the at least one trained model involves in response to determining that an input value resulting from a bone marrow biopsy has been received, analyzing, using a first trained model, the age of the patient together with the plurality of values and the input value. Also, analyzing using the at least one trained model involves in response to determining that an input value resulting from a bone marrow biopsy has not been received, analyzing the age of the patient together with the plurality of values using a second trained model different from the first trained model.

In any aspect provided herein, or embodiments thereof, analyzing the plurality of values each representing a level of a time-varying marker of the plurality of time-varying markers involves analyzing a plurality of values each representing a detected level in a biological sample of a patient of a time-varying marker of the plurality of time-varying markers.

In any aspect provided herein, or embodiments thereof, the method further involves detecting the level of each of the plurality of time-varying markers in the biological sample of the patient, and for a third time-varying marker of the at least one second time-varying marker, comparing levels detected over time in the biological sample of the patient of the third time-varying marker and determining the trajectory value describing the change over time of the third time-varying marker.

In any aspect provided herein, or embodiments thereof, the at least one second time-varying marker contains a third time-varying marker, and the trajectory value describing the change over time of the third time-varying marker indicates whether a value in the biological sample of the patient of the third time-varying marker has increased over time or decreased over time.

In any aspect provided herein, or embodiments thereof, analyzing, with the at least one trained model, the age of the patient together with the plurality of values each representing a level of a time-varying marker of the plurality of time-varying markers involves analyzing, with the at least one trained model: an age of the patient; a free light chain (FLC) ratio for the patient; a level of M-spike for the patient; a level of creatinine for the patient; and a trajectory value indicating whether an amount of hemoglobin increased or decreased.

In any aspect provided herein, or embodiments thereof, the first plurality of numeric values contains a value that is a ratio of detected levels for the patient of two time-varying markers and analyzing the age together with the plurality of values using the at least one trained model involves analyzing the ratio using the at least one trained model.

In any aspect provided herein, or embodiments thereof, the method further involves in response to determining that one of the plurality of values has not been received, determining a value to be used in the analyzing for the one of the plurality of values. In any aspect provided herein, or embodiments thereof, determining the value to be used in the analyzing involves determining the value based on at least one of the plurality of values that were received. In any aspect provided herein, or embodiments thereof, determining the value to be used in the analyzing involves determining the value to be a configured value.

In any aspect provided herein, or embodiments thereof, the analyzing, using the at least one trained model, the age and the plurality of values each representing a level of a time-varying marker involves analyzing the age, the plurality of values, and at least one indicator of whether the patient has been detected to have at least one genetic marker.

In any aspect provided herein, or embodiments thereof, analyzing the age, the plurality of values, and the at least one indicator of whether the patient has been detected to have the at least one genetic marker involves analyzing the age, the plurality of values, and at least one indicator each indicating whether a patient has been found to have a genetic marker selected from one or more of 17 deletion, 17p deletion, 13 deletion, 13q deletion, and 1q gain. In any aspect provided herein, or embodiments thereof, analyzing the age, the plurality of values, and the at least one indicator of whether the patient has been detected to have the at least one genetic marker involves analyzing the age, the plurality of values, and a plurality of indicators each indicating whether a patient has been found to have a corresponding one of 17deletion, 17p deletion, 13 deletion, 13q deletion, and/or 1q gain.

In any aspect provided herein, or embodiments thereof, a time-varying marker is a clinical variable present in an electronic medical record. In any aspect provided herein, or embodiments thereof, serial values for the time-varying marker are annotated at monthly intervals from the date of MGUS or SMM diagnosis.

In any aspect provided herein, or embodiments thereof, the analyzing further involves assessing treatment of the MM precursor disease.

In any aspect provided herein, or embodiments thereof, the multiple myeloma precursor disease progression request contains at least one variable selected from one or more of free light chain (FLC) ratio, M-spike level, age, creatinine level, and hemoglobin level. In any aspect provided herein, or embodiments thereof, the multiple myeloma precursor disease progression request contains two or more of the following variables: free light chain (FLC) ratio, M-spike level, age, creatinine level, and hemoglobin level. In any aspect provided herein, or embodiments thereof, the multiple myeloma precursor disease progression request contains three or more of the following variables: free light chain (FLC) ratio, M-spike level, age, creatinine level, and hemoglobin level. In any aspect provided herein, or embodiments thereof, the multiple myeloma precursor disease progression request contains the following variables: free light chain (FLC) ratio, M-spike level, age, creatinine level, and hemoglobin level.

In any aspect provided herein, or embodiments thereof, the multiple myeloma precursor disease progression request further contains the following variable: bone marrow plasma cell percent (BMPC %).

In any aspect provided herein, or embodiments thereof, the multiple myeloma precursor disease progression request further contains at least one categorical variable selected from one or more of 17 deletion, 17p deletion, 13 deletion, 13q deletion, and 1q gain. Also, the PANGEA machine learning model further contains PANGEA parameters trained for a particular combination of continuous and categorical variables based at least in part on training data. Additionally, the PANGEA machine learning model ingests the at least one variable and the at least one categorical variable to produce the predicted risk throughout the prediction period and render a graphical plot depicting the future risk of progression based on the at least one variable and the at least one categorical variable.

In any aspect provided herein, or embodiments thereof, the at least one categorical variable contains the hemoglobin trajectory.

In any aspect provided herein, or embodiments thereof, the method further involves determining, by the at least one server, a missing variable that is missing from the multiple myeloma precursor disease progression request. The method also further involves determining, by the at least one server, a missing variable prediction machine learning model from a set of missing variable prediction machine learning models based at least in part on the missing variable. Each missing variable prediction machine learning model of the set of missing variable prediction machine learning models contains trained missing variable prediction parameters trained for a particular missing variable based at least in part on missing variable prediction training data. The missing variable prediction training data contains at least one variable value of at least one known variable paired with a known missing variable value of the particular missing variable. The method also further involves utilizing, by the at least one server, the missing variable prediction machine learning model to generate a probability distribution of potential missing variable values for the missing variable based at least in part on the at least one marker measurement of the at least one variable and the trained missing variable prediction parameters. The multiple myeloma precursor disease progression response contains the probability distribution of potential missing variable values, the multiple myeloma precursor disease progression response being configured to cause the computing device to render a graphical plot depicting the probability distribution of potential missing variable values.

The disclosure provides methods (e.g., computer implemented methods) for estimating a risk that a condition of a patient with a multiple myeloma (MM) precursor disease such as MGUS or SMM will progress into MM. Compositions and articles defined by the disclosure were isolated or otherwise manufactured in connection with the examples provided below. Other features and advantages of the disclosure will be apparent from the detailed description, and from the claims.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, nucleic acid molecule, polypeptide, or fragments thereof.

By “alteration” is meant a change in the structure, expression levels or activity of a marker or clinical variable as detected by standard art known methods such as those described herein. The alteration can be an increase or a decrease. As used herein, an alteration includes a 10%, 20%, 30%, 40%, 50%, 60%, 70%, 80%, 90% or greater change in the level of the marker or clinical variable.

By “biological sample” is meant a sample obtained from a subject. In some embodiments, a biological sample is a blood, sera, plasma, or bone marrow sample.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

“Detect” refers to identifying the presence, absence or amount of an analyte to be detected.

By “disease” is meant any condition or disorder that damages or interferes with the normal function of a cell, tissue, or organ. Examples of diseases include multiple myeloma (MM) and MM precursor diseases, such as smoldering multiple myeloma (SMM) and monoclonal gammopathy of undetermined significance (MGUS).

By “effective amount” is meant the amount of an agent required to ameliorate the symptoms of a disease relative to an untreated patient. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In an embodiment, an effective amount is the amount required to stabilize or slow the progression of a MM precursor disease.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

By “increase” is meant to alter positively relative to a reference. An increase may be by 1%, 5%, 10%, 25%, 30%, 50%, 75%, 100%, or more, or by 1.5-fold, 2-fold, 3-fold, 4-fold, 5-fold, 6-fold, 7-fold, 8-fold, 9-fold, 10-fold, 25-fold, 50-fold, 75-fold, 100-fold, or more.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, and or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant a clinical variable that is associated with a disease or disorder. In some embodiments, the marker is a polypeptide, polynucleotide or other analyte whose level is detected during the characterization of a disease (e.g., MM precursor disease or MM). In some embodiments, an alteration in a marker's expression level, concentration, abundance, activity or structure is detected. In some embodiments, an alteration in a marker is detected over time (e.g., over days, weeks, months, years). In embodiments, the marker is a genetic marker, such as a 17 deletion, 17p deletion, 13 deletion, 13q deletion, and/or 1q gain. In some embodiments, the marker is a free light chain (FLC), an M protein, creatinine, hemoglobin, and/or a bone marrow plasma cell.

By “genetic marker” is meant a polynucleotide sequence within a genome having an alteration associated with a developmental state, condition, disease, or disorder. In embodiments, the genetic marker is a 17 deletion, 17p deletion, 13 deletion, 13q deletion, and/or 1q gain.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the disclosure embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant to alter negatively relative to a reference. A reduction may be by 1%, 5%, 10%, 25%, 30%, 50%, 75%, 100%, or more, or by 1.5-fold, 2-fold, 3-fold, 4-fold, 5-fold, 6-fold, 7-fold, 8-fold, 9-fold, 10-fold, 25-fold, 50-fold, 75-fold, 100-fold, or more.

By “reference” is meant a standard or control condition. Non-limiting examples of references include healthy cells, cells associated with multiple myeloma (MM), soldering multiple myeloma (SMM), or monoclonal gammopathy of undetermined significance (MGUS), such as cells obtained from a patient with MM, SMM, or MGUS. In embodiments, a reference is a healthy subject or a subject with MM, SMM, or MGUS. In some cases, a reference is a subject administered an agent, a subject prior to a change in treatment, a subject prior to treatment and/or administration of an agent, a subject not administered an agent, a subject being administered a treatment, or a subject having completed a course of treatment.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween.

By “specifically binds” is meant a compound or antibody that recognizes and binds a polypeptide of the disclosure, but which does not substantially recognize and bind other molecules in a sample, for example, a biological sample, which naturally includes a polypeptide of the disclosure.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule.

By “hybridize” is meant to pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, about less than about 500 mM NaCl and 50 mM trisodium citrate, or about less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., of at least about 42° C., or of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. In embodiments, a treatment involves one or more of characterizing a biological sample and/or a neoplasia, monitoring a patient and/or neoplasia in the patient, and prognosis.

Unless specifically stated or obvious from context, as used herein, the term “or” is understood to be inclusive. Unless specifically stated or obvious from context, as used herein, the terms “a”, “an”, and “the” are understood to be singular or plural.

Use of ordinal terms such as “first,” “second,” “third,” etc., in the claims to modify a claim element does not by itself connote any priority, precedence, or order of one claim element over another or the temporal order in which acts of a method are performed but are used merely as labels to distinguish one claim element having a certain name from another element having a same name (but for use of the ordinal term) to distinguish the claim elements.

Unless specifically stated or obvious from context, as used herein, the term “about” is understood as within a range of normal tolerance in the art. In some cases, a range of normal tolerance in the art is within 1 or 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides a flow chart showing a description of the training and validation cohorts of the PANGEA Project. Both cohorts comprise monoclonal gammopathy of undetermined significance (MGUS) and smoldering multiple myeloma (SMM) patients. In FIG. 1, “BM” indicates a bone marrow sample was taken.

FIGS. 2A and 2B provide Forest plots of clinical variables conferring precursor progression risk within the PANGEA Models. FIG. 2A provides a Forest plot for the PANGEA (BM) Model demonstrating risk predicted by involved-to-uninvolved free light chain (FLC) ratio, M-spike, age, creatinine, bone marrow plasma cell percent (BMPC %), and a trajectory for decrease in hemoglobin. FIG. 2B provides a Forest plot for the PANGEA (No BM) Model demonstrating risk predicted by FLC ratio, M-spike, age, creatinine, and a trajectory for decrease in hemoglobin.

FIGS. 3A and 3B provide Kaplan-Meier curves of time to progression in Validation Cohort 1 as predicted by the (FIG. 3A) PANGEA (BM) Model and (FIG. 3B) PANGEA (No BM) Model. Patients were divided into four groups based on quartiles around the mean as indicated by the risk categories of Low Risk, Intermediate-Low Risk, Intermediate-High Risk, and High Risk.

FIGS. 4A and 4B provide Kaplan-Meier curves of time to progression in Validation Cohort 2 as predicted by the (FIG. 4A) PANGEA (BM) Model and (FIG. 4B) PANGEA (No BM) Model. Patients were divided into four groups based on quartiles around the mean as indicated by the risk categories of Low Risk, Intermediate-Low Risk, Intermediate-High Risk, and High Risk.

FIGS. 5A-5D provide plots showing risk stratification of Validation Cohort 1 at first visit for (FIG. 5A) all smoldering multiple myeloma (SMM) patients or (FIG. 5B) SMM patients who progressed to MM, by the PANGEA (BM) Model compared to the Rolling 20/2/20 Model and risk stratification of Validation Cohort 1 at Visit 1 for (FIG. 5C) all SMM patients and (FIG. 5D) SMM patients who progressed to MM, by the PANGEA (No BM) Model compared to the Rolling 20/2/20 Model.

FIGS. 6A-6D provide plots showing progression-free survival (PFS) of all smoldering multiple myeloma (SMM) patients in the PANGEA Project (Training Cohort, Validation Cohort 1, Validation Cohort 2). Kaplan-Meier curves indicating (FIG. 6A) PFS of PANGEA SMM patients, (FIG. 6B) PFS of patients who progressed from SMM to MM vs. non-progressors, (FIG. 6C) PFS for SMM patients stratified by 20/2/20 risk group, and (FIG. 6D) PFS for SMM patients stratified by 20/2/20 number of risk factors with separation of the high-risk group into patients with 2 risk factors vs. 3 risk factors.

FIGS. 7A and 7B provide plots showing progression-free survival (PFS) of all monoclonal gammopathy of undetermined significance (MGUS) patients in the PANGEA Project (Training Cohort, Validation Cohort 1, Validation Cohort 2). FIG. 7A provides a Kaplan-Meier plot of PFS for all patients with MGUS diagnosis. FIG. 7B provides a plot showing PFS for all International Myeloma Working Group (IMWG) patients stratified by IMWG risk criteria.

FIGS. 8A and 8B provide box-and-whisker plots showing bootstrapping (C-Statistic and confidence intervals) for Validation Cohort 1 comparing the Rolling 20/2/20 Model and (FIG. 8A) PANGEA (BM) Model, (FIG. 8B) PANGEA (No BM) Model.

FIG. 9 provides a Forest plot of risk factors associated with progression in the PANGEA Model (FISH) in a subcohort of patients who had FISH testing available.

FIG. 10 is a block diagram of a system with which some embodiments may operate.

FIG. 11 is a flowchart of a process that may be implemented in some embodiments to evaluate a patient's risk of MM disease progression.

FIG. 12 is a block diagram of a computing device with which some embodiments may operate.

FIGS. 13A-13C provide plots showing trended hemoglobin of the PANGEA Project for (FIG. 13A) MGUS and SMM patients, (FIG. 13B) male and female patients, and (FIG. 13C) PANGEA risk groups.

FIGS. 14A-14D provides plots showing risk stratification of Validation Cohort 2 at First Visit for (FIG. 14A) all MGUS patients and (FIG. 14B) MGUS patients who progressed to MM, by the PANGEA (BM) Model compared to the Rolling IMWG Model and risk stratification of Validation Cohort 1 at First Visit for (FIG. 14C) all MGUS patients and (FIG. 14D) MGUS patients who progressed to MM, by the PANGEA (No BM) Model compared to the IMWG Rolling Model.

FIG. 15 provides a plot showing regression residual for the variable “age” of the PANGEA Model (BM) to investigate potential variations of the effects of age on the risk of progression over time.

FIG. 16 provides a plot showing the effect of the predictor “bone marrow plasma cell percentage” on the risk of progression. The plot illustrates the effects, estimated with a survival model that utilizes splines to capture the relation (log of relative risk) between the biomarker and the risk of progression. This is considered an extension f (x) of the linear relation of the PANGEA Model.

FIGS. 17A and 17B provide plots showing PANGEA Model calibration in three independent datasets for the (FIG. 17A) PANGEA Model (No BM) and (FIG. 17B) PANGEA Model (BM). Illustrated are the expected number of events and actual number of events at 1, 2, 3, and 5 years after Visit 1.

### DETAILED DESCRIPTION OF THE DISCLOSURE

Described herein are techniques for estimating a risk that a condition of a patient with a multiple myeloma (MM) precursor disease such as MGUS or SMM will progress into MM, such as a risk that the patient's condition will progress into MM within a timeframe or across multiple timeframes (e.g., successive time frames).

In some embodiments described herein, information regarding several markers is curated in specific ways and an estimate of risk is produced based on analyzing that specifically-curated information with at least one trained model. These markers may be or include time-varying markers. For example, in some embodiments, an age of the patient may be analyzed by the trained model(s) together with multiple values each corresponding to a time-varying marker for the patient. The multiple values may include numeric values (e.g., continuous values, such as values on a continuous numeric scale) for a value of one or more time-varying markers at a time a sample was obtained, such as a blood sample for the patient. In some such embodiments, the multiple values may also include a trajectory value for one or more other time-varying markers, where the trajectory value may be a binary value or other qualitative indication of whether a value of a time-varying marker has been increasing or decreasing over time, or has been holding steady over time, or other indication of a change over time of the time-varying marker. In some embodiments described herein, this combination of age, quantitative/numeric values for some markers, and qualitative/trajectory values for other markers may be analyzed with the trained model(s) and a value indicating risk that the patient's condition will progress into MM may be output. The value indicating risk may be a quantitative value specific to the patient and may indicate a risk for the patient over a timeframe. In some embodiments, multiple numeric values may be output, where each value indicates a risk for the patient that the patient's condition will progress to MM within a corresponding timeframe, where the timeframes may be successive time periods.

While multiple myeloma (MM) is always preceded by two precursor conditions, clinicians and patients have long struggled to identify the chances that a patient's condition will progress from one precursor condition into the next or into MM. Only a fraction of patients with MGUS will ever have their condition progress to SMM, and only a fraction of patients with SMM will have their condition progress into MM. A chief difficulty for a patient with a precursor condition is thus the uncertainty of the patient's prospects of their condition developing into a rare and serious cancer. The uncertainty of whether MM will develop is compounded by uncertainty of whether and when MGUS could develop into SMM, or whether and when SMM could develop into MM. The ambiguity surrounding the patient's future condition and timeline for that condition creates stress for the patient and their family and friends and creates difficulties for clinicians in managing the patient's care and the patient's expectations.

Given the severe disadvantages associated with the lack of predictability for the condition of a patient with an MM precursor condition, researchers and clinicians have long investigated MM precursor conditions and sought signs and signals that an MM precursor condition could progress or a timeline on which it will progress. But while countless hours/years and resources have been invested in this research and multiple techniques have been developed, a reliable manner of estimating a patient's risk has not been identified. The current best-available technique, while an improvement on previous approaches, still fares little better than a coin flip (i.e., a C-statistic of 0.530) in terms of predictive reliability for disease progression.

This best-available technique, the “20/2/20” system developed by the Mayo Clinic and updated by the International Myeloma Working Group (IMWG), focuses on stratifying a patient into one of a set of three risk categories. The risk category for a patient is selected based on whether the patient has 0, 1, or 2+ risk factors, from among the system's risk factors. The system is termed “20/2/20” based on three such risk factors: (1) whether or not a patient's free light chain (FLC) ratio is greater than 20; (2) whether or not the patient's monoclonal protein level is greater than 2.0 g/dL; and (3) whether or not the patient's bone marrow plasma cell (BMPC) percentage is greater than 20%. Additional risk factors include whether certain genetic factors are present, such as fluorescence in-situ hybridization (FISH) indicating presence of translocation t4;14); presence or absence of translocation t(14;16); whether or not the patient has a gain in chromosome 1q; and whether or not the patient has a deletion in chromosome 13/13q. Each of these factors is considered on a binary yes/no basis, and the number of “yes” factors is totaled. If the patient has 0 risk factors, the patient is considered low-risk for disease progression. One risk factor is intermediate-risk and 2+ risk factors is high-risk.

In addition to the unreliability of this technique, the inventors have recognized drawbacks to the manner in which the “20/2/20” system's analysis is structured and that serve to undermine its reliability and its usefulness.

Among these drawbacks is the categorization/stratification on which the conventional system relies. As discussed above, the system places patients into low, intermediate, and high risk categories based on the number of detected risk factors for the patient. The patient's risk of precursor disease progression is thus reported based on risk associated with a population of patients that have the same number of risk factors, without link to what that patient's specific risk factors are. The risk that is reported is thus not an individualized risk for the patient and is not based on the patient's own condition. This limits the reliability of the conventional risk determination.

The inventors have recognized and appreciated that one approach to generation of individual risk assessments is use of trained machine learning models. For example, a machine learning model may be configured to analyze certain features and be provided during a training phase with prior patient information including values for those features and patient outcome information. The model may be trained to associate feature values and outcomes, and once trained may be provided with those same features for a different patient and output a risk assessment for that patient's disease. The inventors have also recognized and appreciated the reliability of a machine learning model at least partially depends on the accuracy and quality of the data used to train the model.

The inventors have further recognized and appreciated that the “20/2/20” model is disadvantageous because it requires painful and invasive testing. One of the risk factors on which the “20/2/20” system relies is BMPC %, which can only be determined by obtaining a bone marrow biopsy. Such a biopsy requires a clinician drill into the patient's bone and sample marrow for testing. For the biopsy to be performed, a patient must visit a medical facility for an invasive procedure that may require administration of at least a local anesthetic and potentially a team of clinicians to perform the procedure, after which other clinicians may evaluate the procedure and report back. The procedure is painful for the patient as well as likely to be inconvenient, particularly when a patient is subject to repeat testing for condition monitoring purposes, and high-cost to the clinicians and the payor (either the patient or another). Moreover, such biopsies are subject to inconsistencies that undermine their utility. For example, a location at which the biopsy is taken from bone may impact the plasma cell percentage, with higher or lower values stemming from location of the biopsy rather than the patient's condition. The plasma cell percentage may also vary over time and over a patient's recent circumstances, meaning that a timing of the biopsy may impact the clinical determination made from it. And such clinical determinations may vary based on the pathologist or pathology tool drawing those conclusions, as interpretation differences may yield different clinical determinations. Despite all this, the biopsy value is still used and, indeed, is a primary factor used in the conventional approach. It would be advantageous for patients, clinicians, payors, and others if a risk estimation system could be developed that relies, at least in part, on information that could be obtained from a sample of a patient's fluids, such as a sample of the patient's blood, that can be obtained less invasively and with less complexity than a bone marrow biopsy.

The inventors have also recognized and appreciated that the “20/2/20” system is hampered by its pure reliance on qualitative assessment of each risk factor, including in fixed analyses where numeric values for marker levels are considered to yield yes/no answers to presence of a risk factor. As discussed above, the “20/2/20” name comes from the fixed thresholds by which three risk factors are judged. The inventors have recognized and appreciated that the use of fixed thresholds reflects an assumption regarding the values that are judged by these thresholds that is prone to error. The use of a fixed threshold to guide whether a marker is suggesting increased risk indicates that the marker remains at a relatively consistent level and that when a value for the marker is detected to be above the fixed threshold, that indicates an evolving and increasing risk. But the inventors have recognized and appreciated that informative markers for MM risk do not have levels that remain consistent, but instead naturally vary up and down over time. Such a time-varying marker could have a higher or lower value within a short time span, depending on circumstances or times at which samples for the marker were taken.

Beyond recognizing and appreciating the advantages of a risk analysis that operates with time-varying markers, the inventors have further recognized the advantages of specific ways in which to analyze those markers. The inventors have recognized and appreciated that other forms of analysis are informative for time-varying thresholds, in addition to comparison to a fixed threshold, and further recognized and appreciated that a combination of analyses may be particularly informative. Calculating risk based on a numeric value indicating a level of a marker for a patient may be one useful form of analysis. In some cases, such a numeric value may be from a non-integer numeric scale, such as a continuous numeric scale. As another example, analyzing a change in a time-varying marker may be advantageous. Such a change in a time-varying marker may be a rate of change in a value indicating a level of a marker, such as a change over time in the marker. As another example, such a change may be a direction of a change, such as a trajectory of the marker. Such a trajectory may indicate whether a level of the marker is increasing or decreasing, or increasing, decreasing, or staying consistent. In some embodiments, trajectory may be measured over a recent time period, such as over the past day, past week, past month, past three or six months, past year, or past five years, or other suitable time period. In embodiments that consider whether a level is staying consistent, a determination may be made of whether a value has changed more than a threshold amount (an absolute threshold or a relative threshold, such as a percentage change). The inventors have recognized and appreciated that while there are a number of useful analyses that can be performed in connection with time-varying markers, a particularly advantageous risk estimation system would leverage a combination of analyses, such as by analyzing numeric values for some markers and change indications (e.g., trajectory values) for others. Accordingly, an advantageous system for estimation of MM precursor disease progression would not only leverage time-varying markers but would also leverage a combination of quantitative and qualitative information regarding such markers, where the qualitative information may include information regarding a change in a time-varying marker.

The inventors have thus recognized that determining an individual risk for a patient that that patient's condition will progress from an MM precursor disease into another precursor disease or into MM would be advantageous and could increase reliability of the metric. But beyond that, the inventors have also recognized and appreciated that a risk estimation technique that leverages the dynamic nature of a patient's markers, including time-varying markers and quantitative information regarding some markers and qualitative information regarding change of other markers, would be desirable and may yield increased accuracy. The inventors have further recognized and appreciated that if such time-varying markers could be determined from a sample of a patient's fluids, such as a patient's blood, this would increase the convenience, reduce the burdens, and improve the cost of testing for a patient.

Described herein are techniques for using a combination of time-varying markers for a patient in connection with a trained model, to generate a value indicating a risk that a condition of a patient with an MM precursor disease will progress to another MM precursor disease or to MM. In some embodiments described herein, an estimate of a patient's risk of MM disease progression is generated based on analyzing specifically-curated information for time-varying markers with at least one trained model. For example, in some embodiments, an age of the patient may be analyzed by the trained model(s) together with multiple values each corresponding to a time-varying marker for the patient. The multiple values may include numeric values for a value of one or more time-varying markers at a time a sample was obtained, such as a blood sample for the patient. In some such embodiments, the multiple values may also include a trajectory value for one or more other time-varying markers, where the trajectory value may be a binary value or other qualitative indication of whether a value of a time-varying marker has been increasing or decreasing over time, or has been holding steady over time, or other indication of a change over time of the time-varying marker. In some embodiments described herein, this combination of age, quantitative/numeric values for some markers, and qualitative/trajectory values for other markers may be analyzed with the trained model(s) and a value indicating risk that the patient's condition will progress into MM may be output. The value indicating risk may be a quantitative value specific to the patient and may indicate a risk for the patient over a timeframe. In some embodiments, multiple numeric values may be output, where each value indicates a risk for the patient that the patient's condition will progress to MM within a corresponding timeframe, where the timeframes may be successive time periods.

### Hematological Malignancies

Multiple myeloma (MM) is a plasma cell dyscrasia. Plasma cell dyscrasias are cancers of the plasma cells. They are produced as a result of malignant proliferation of a monoclonal population of plasma cells that may or may not secrete detectable levels of a monoclonal immunoglobulin or paraprotein commonly referred to as M protein. Further non-limiting examples of plasma cell dyscrasias include monoclonal gammopathy of undetermined significance (MGUS), smoldering multiple myeloma (SMM), symptomatic multiple myeloma, Waldenstrom macroglobulinemia (WM), amyloidosis (AL), plasmacytoma syndrome (e.g., solitary plasmacytoma of bone, extramedullary plasmacytoma), light chain deposition disease, and heavy-chain disease. MGUS, SMM, and symptomatic MM represent a spectrum of the same disease.

MGUS is characterized by a serum monoclonal protein (<30 g/L), <10% plasma cells in the bone marrow, and absence of end-organ damage. Asymptomatic MGUS stage consistently precedes multiple myeloma (MM). MGUS is present in 3% of persons >50 years and in 5%>70 years of age. The average risk of progression to MM or a related disorder is 1% per year. Patients with risk factors consisting of an abnormal serum free light chain ratio, non-immunoglobulin G (IgG) MGUS, and an elevated serum M protein (≥15 g/l) have a risk of progression at 20 years of 58%, compared with 37% among patients with two risk factors, 21% for those with one risk factor, and 5% for individuals with no risk factors. The cumulative probability of progression to active MM or amyloidosis is 51% at 5 years, 66% at 10 years and 73% at 15 years; the median time to progression was 4.8 years

Smoldering Multiple Myeloma (SMM) also known as asymptomatic MM is characterized by having a serum immunoglobulin (Ig) G or IgA monoclonal protein of 30 g/L or higher and/or 10% or more plasma cells in the bone marrow but no evidence of end-organ damage.

Symptomatic or Active Multiple Myeloma (MM) is a form of cancer that affects a type of white blood cell called the plasma cell. Multiple myeloma appears in the bone marrow, which is the soft tissue inside the bones that makes stem and immune cells. In multiple myeloma, plasma cells, which mature from stem cells and typically produce antibodies to fight germs and other harmful substances, become abnormal. These abnormal cells are called myeloma cells. In 2021, an estimated 34,920 cases of multiple myeloma were diagnosed in the United States and over 12,410 patient deaths associated with multiple myeloma were reported. As the most common type of plasma cell cancer, effective treatment requires an accurate diagnosis and precise treatment.

Symptomatic or active MM is characterized by any level of monoclonal protein and the presence of end-organ damage that consists of the SLIM-CRAB criteria (bone marrow plasma cell percentage ≥60%, involved: uninvolved free light chain ratio>100, >1 focal lesion on MRI, hypercalcemia, renal insufficiency, anemia, or bone lesions). MM is a plasma cell malignancy that characteristically involves extensive infiltration of bone marrow (BM), with the formation of plasmacytomas, as clusters of malignant plasma cells inside or outside of the BM milieu. Consequences of this disease are numerous and involve multiple organ systems. Disruption of BM and normal plasma cell function leads to anemia, leukopenia, hypogammaglobulinemia, and thrombocytopenia, which variously result in fatigue, increased susceptibility to infection, and, less commonly, increased tendency to bleed. Disease involvement in bone creates osteolytic lesions, produces bone pain, and may be associated with hypercalcemia.

### Conventional Detection Methods

To date, the gold standard for characterizing MM disease state has involved a bone marrow biopsy. In embodiments, the present disclosure provides a non-invasive method for characterizing the disease state of a patient. The methods of the disclosure are suitable for use alone, or if desired, may be used in concert with one or more of the following conventional diagnostic methods.

Traditionally, the initial evaluation of a suspected hematological malignancy (e.g., a monoclonal gammopathy) includes both serum and urine protein electrophoresis with immunofixation to identify and quantify the M protein. The majority of patients are expected to have a detectable M protein, but approximately 1-3% can present with a non-secretory myeloma that does not produce light or heavy chains. True non-secretory myeloma is thus rare, not least because, with the availability of serum free light chain testing and new mass spectrometry techniques (which are more sensitive but less often used than serum protein electrophoresis), it is recognized that M protein is present. The most common M protein is IgG, followed by IgA, and light chain-only disease. IgD and IgE are relatively uncommon and can be more difficult to diagnose because their M spikes are often very small. The present disclosure provides methods that can also be used to characterize a monoclonal gammopathy in a patient.

A standard evaluation of a documented monoclonal gammopathy includes a complete blood count with differential, calcium, serum urea nitrogen, LDH, beta-2 microglobulin, creatinine and urinalysis. Serum free light chain testing is also a useful diagnostic test (Piehler A. P. et al, Clin. Chem., 54:1823-30 (2008)). Bone disease is best assessed by skeletal survey. Bone scans are not a sensitive measure of myelomatous bone lesions because the radioisotope is poorly taken up by lytic lesions in MM, as a result of osteoblast inhibition. Magnetic resonance imaging (MRI) is useful for the evaluation of solitary plasmacytoma of bone and for the evaluation of paraspinal and epidural components. 18F-FDG Positron Emission Tomography (PET)/CT scans are more sensitive in the detection of active lesions in the whole body (Fonti R. et al., J. Nucl. Med., 49: 195-200 (2008)). A bone marrow aspiration and biopsy are helpful to quantify the plasma cell infiltrate and adds important prognostic information with cytogenetic evaluation, including fluorescent in situ hybridization (FISH).

The criteria for the diagnosis of MM, SMM, and MGUS are detailed in Table A below. Distinction among these disease states informs treatment decisions and prognostic recommendations.

TABLE A
Conventional criteria for the diagnosis of MM, SMM, and MGUS

| Disorder | Disease definition |
|---|---|
| MGUS | Serum monoclonal protein level <3 g/dL, |
| | bone marrow plasma cells 10%, |
| | and absence of end-organ damage, such |
| | as lytic bone lesions, anemia, |
| | hypercalcemia, or renal failure, that |
| | can be attributed to a plasma cell |
| | proliferative disorder. |
| SMM | Serum monoclonal protein (IgG or IgA) |
| | level ≥3 g/dL and/or bone marrow |
| | plasma cells ≥10%, absence of end-organ |
| | damage, such as lytic bone |
| | lesions, anemia, hypercalcemia, or |
| | renal failure that can be attributed to a |
| | plasma cell proliferative disorder. |
| | Alternatively, or additionally, SMM |
| | may be defined by the absence of |
| | markers (e.g., Myeloma Defining Event |
| | markers), by bone-marrow plasma |
| | cell infiltration ε60%, by serum-free |
| | light chain ratio ε100, and/or by >1 |
| | focal lesion in the skeleton on magnetic |
| | resonance imaging analysis (see, |
| | Rajkumar SV, et al. International Myeloma |
| | Working Group updated criteria |
| | for the diagnosis of multiple myeloma. |
| | Lancet Oncol 15, e538-548 (2014)). |
| | In some instances, SMM may be associated |
| | with organ damage. |
| MM | Bone marrow plasma cells ≥10%, |
| | presence of serum and/or urinary |
| | monoclonal protein (except in patients |
| | with true nonsecretory multiple |
| | myeloma), plus evidence of lytic bone |
| | lesions, anemia, hypercalcemia, or |
| | renal failure that can be attributed to the |
| | underlying plasma cell proliferative |
| | disorder. |

Conventional staging systems involve the following. The most widely used myeloma staging system since 1975 has been the Durie-Salmon, in which the clinical stage of disease is based on several measurements including levels of serum M protein, serum hemoglobin value, serum calcium level, and the number of bone lesions. The International Staging System (ISS), developed by the International Myeloma Working Group is now also widely used (Greipp P R. Et al, J. Clin. Oncol, 23:3412-20 (2005)). ISS is based on two prognostic factors: serum levels of B2M and albumin and is comprised of three stages: B2M 3.5 mg/L and albumin 3.5 g/dL (median survival, 62 months; stage I); B2M <3.5 mg/L and albumin <3.5 g/dL or B2M 3.5 to <5.5 mg/L (median survival, 44 months; stage II); and B2M 5.5 mg/L (median survival, 29 months; stage III). With an increased understanding of the biology of myeloma, other factors have been shown to correlate well with clinical outcome and are now commonly used. For example, cytogenetic abnormalities as detected by FISH techniques have been shown to identify patient populations with very different outcomes. For instance, loss of the long arm of chromosome 13 is found in up to 50% of patients and, when detected by metaphase chromosome analysis, is associated with poor prognosis. In addition, t(4;14), and −17p13 is typically associated with poor outcome, while t(11;14) and hyper diploidy are associated with improved survival (Kyrtsonis M. C. et al., Semin. Hematol, 46:110-7, (2009)).

### Precursor Asymptomatic Neoplasms by Group Effort Analysis (PANGEA) Model

In various aspects, the disclosure provides a model termed the “Precursor Asymptomatic Neoplasms by Group Effort Analysis (PANGEA) Model.” In some aspects, the disclosure provides an online calculator implementing the PANGEA Model that allows clinicians and patients to assess individual risk of progression and consider early therapeutic interceptions.

In one embodiment, the PANGEA model was developed, as described further in the Examples, based upon a cohort of patients with longitudinal data who were diagnosed with MGUS or SMM at baseline. Using these data, a model was developed termed the “PANGEA Model.” In some cases, the model is a multivariate Cox model with time-varying and trajectory markers with or without bone marrow data to estimate progression risk. A PANGEA Model can be validated, as described in the Examples, in independent cohorts of patients from various regions, such as Greece, the United Kingdom, and the Czech Republic.

In embodiments, the PANGEA Model uses time-varying markers (e.g., monoclonal protein, free light chain, age, creatinine, bone marrow plasma cell percentage) and dynamic trajectories (e.g., hemoglobin) to predict progression from precursor disease (e.g., MGUS or SMM) to MM. In embodiments, the PANGEA Model outperforms the 20/2/20 model with and/or without bone marrow data with an improvement in C-statistic of about or at least about 5%, 10%, 15%, 20%, 25%, 30%, 35%, 40%, 45%, 50%, 55%, 60%, 65%, 70%, 75%, 80%, 85%, 90%, 95%, 100%, or more. In embodiments, the models provided herein outperform previous models in multiple cohorts of patients. Therefore, in embodiments, the models provided herein improve precursor progression predictions using variables available to all clinicians and, regardless of bone marrow data, improve upon current MGUS/SMM risk stratification systems. In some instances, the models provided herein account for increased progression risk observed in patients with 1q gain, 13/13q deletion, or 17/17p deletion, and/or MYC alterations.

In some cases, the PANGEA Model uses time-varying clinical markers without arbitrary cutoffs to model how precursor progression risk to MM evolves for a single patient over time, regardless of whether a bone marrow biopsy (BMbx) is conducted.

In embodiments, the methods provided herein are used for the identification of individual patients with precursor myeloma diseases (MGUS and SMM) who are at the highest risks of disease progression to overt myeloma so that treatment can intercept disease early and improve prognosis.

In embodiments, the PANGEA Model eliminates the need for serial bone marrow biopsies. As described herein, the 20/2/20 criteria, a gold standard for smoldering multiple myeloma (SMM) disease progression prediction, demonstrate a c-statistic of 0.530 with 0.50 indicative of a 50% chance (or coin flip) of accurately predicting disease progression risk. In various instances, the PANGEA Model improves upon this risk prediction metric by >20% and thus vastly increases clinical abilities to identify high-risk patients. The 20/2/20 criteria stratify patients into three risk groups with three risk probabilities. Advantageously, the PANGEA Model can be applied to individual patients and, therefore, give an individual patient their own, personalized disease progression risk. In embodiments, these benefits are all built directly into an online web application which prompts for simple inputs that can be readily used by both clinicians and patients.

The PANGEA Model can be used to compute individual multiple myeloma (MM) progression probabilities for patients that have precursor conditions. As a result, it can be used to stratify patients based on their individual predicted risk. This allows for clustering of patients at higher risk of disease progression and selection of patient populations for the development of precursor clinical trials, both by medical centers and for-profit organizations. Also, in embodiments, the model can be routinely used by physicians to decide the appropriate time intervals for individual patient follow-up.

In some cases, the PANGEA Model is a useful tool that leverages machine learning (ML) techniques in the MM setting. In some instances, the PANGEA Model improves on the 20/2/20 Baseline and Rolling Models with an increase in validation cohort C-statistic at 1st, 2nd, and 3rd patient visit. In some cases, the PANGEA Model (BM) and PANGEA Model (No BM) outperform the Baseline and Rolling Models, as described in the Examples. C-statistics can be used as measures of predictability for current and new models with model improvement indicated by a change in c-statistic greater than about or at least about 1%, 5%, 10%, 15%, 20%, 25%, 30%, 35%, 40%, 45%, 50%, 55%, 60%, 65%, 70%, 75%, 80%, 85%, 90%, 95%, 100%, or more. In embodiments, the models provided herein have c-statistic increase of greater than about or at least about 1%, 5%, 10%, 15%, 20%, 25%, 30%, 35%, 40%, 45%, 50%, 55%, 60%, 65%, 70%, 75%, 80%, 85%, 90%, 95%, 100%, or more with respect to a baseline model (e.g., a 20/2/20 model (baseline and/or dynamic)). The methods provided herein provide for improvements over current clinical standards in predicting progression from SMM to MM disease.

In some instances, the PANGEA Model accounts for genomic and circulating tumor cell data.

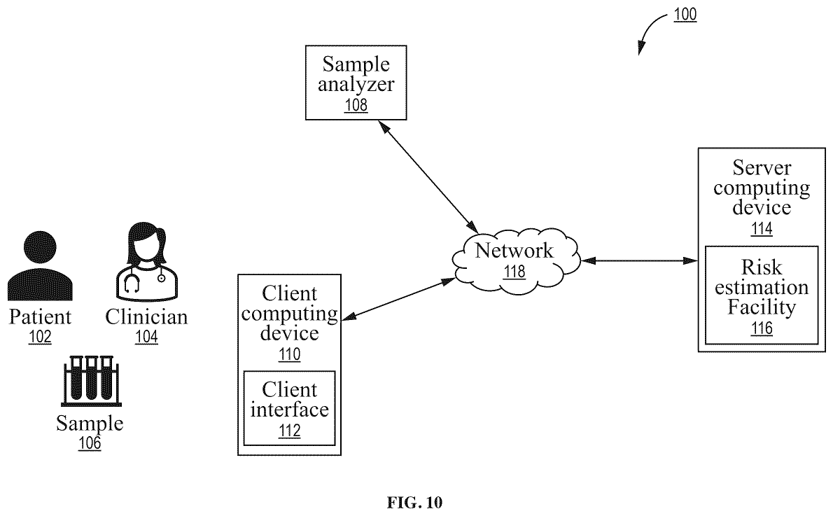

FIG. 10 illustrates a block diagram of a system 100 with which some embodiments may operate. The system 100 can estimate a risk that a condition of a patient with a multiple myeloma (MM) precursor disease such as MGUS or SMM will progress into MM, such as a risk that the patient's condition will progress into MM within a timeframe or across multiple timeframes (e.g., successive time frames). The system 100 may in some embodiments produce the estimate of risk by analyzing a combination of time-varying markers with a trained model. These markers may be or include time-varying markers.

The system 100 can include a patient 102. In some embodiments, the patient 102 has a multiple myeloma (MM) precursor disease. For example, the patient 102 can be a recipient of health care services that are administered by healthcare professionals. For example, the patient 102 can be ill or injured and require treatment. The patient 102 can seek the advice of healthcare professionals regarding treatment, which may, for example, be a response to pain or a feeling of unease. Accordingly, one or more clinicians 104 may interface with the patient 102 to manage illness or injury of the patient. Examples of clinicians 104 include a physician, nurse, physician assistant, nurse practitioner, psychologist, clinical pharmacist, clinical scientist, or specialist physician such as a pathologist.

One or more samples 106 may be obtained from the patient 102, such as by the clinician 104 obtaining the sample through drawing fluid, tissue, or other material from the patient 102. In some embodiments, obtaining fluid may include taking a blood sample from the patient 102 and obtaining tissue may include performing a bone marrow biopsy from the patient 102, though it should be appreciated that embodiments are not so limited. In some embodiments, the sample 106 may be sufficient to produce genetic data for the patient 102, such as through analyzing the fluid, tissue, or other material for genetic information. More generally, the sample 106 can be gathered from the body to aid in medical diagnostics or evaluation of treatment. The sample 106 can include matter from blood, solid tissue, soft tissue, bodily fluids, or any other matter described herein or that could be leveraged to obtain the types of patient information (e.g., time varying markers) described herein. Means of gathering the sample 106 can include normal excretion, biopsy, and excision or swabbing. The sample 106 can be treated prior to use, which can include culturing or preservation for transport.

The system 100 can include a sample analyzer 108, which may be or include one or more tools for analyzing the sample 106. The exact form of the sample analyzer 108 may depend on the form of the sample(s) 106 taken from the patient. Examples of analytical tools for detecting markers were discussed above, any one, two, or more of which may be implemented in some embodiments. In some embodiments, the sample analyzer 108 may include a blood analyzer and/or a bone marrow biopsy analyzer. In some embodiments in which the sample analyzer 108 includes a blood analyzer, analyzing the blood of the sample 106 from the patient 102 may include determining the chemical composition of blood in the sample 106, which may include screening for the presence of an analyte of interest or disease biomarker.

The sample analyzer 108 can be used at the point of care, such as a clinic, or in a laboratory, such as in a hematology laboratory. The sample analyzer 108 can include specialized instrumentation for high-specificity or high-resolution output, such an immunoassay analyzer, or may involve lower resolution point-of-care diagnostics, such as pH strips for pH testing.

The system 100 can include a client computing device 110, which may be a desktop or laptop personal computer, smart mobile phone, server, or other suitable device. The client computing device 110 may include a client interface 112 by which the patient 102 or the clinician 104 may interact with the client computing device 110. For example, the patient 102 or the clinician 104 can use the client interface 112 to interface with the sample analyzer 108 or risk estimation facility 116 of the server computing device 114. For example, the patient 102 and/or clinician 104 may operate the client interface 112 to initiate analysis of the sample 106 by the sample analyzer 108 and display analysis results such as whether markers were detected and/or levels of those markers in the client interface 112. The patient 102 and/or clinician 104 may additionally or alternatively operate the client interface 112 to input markers and/or marker levels obtained from the sample analyzer 108, such as output to the patient 102 and/or clinician 104 in another interface. Those values may be provided to the risk estimation facility 116. As a further example, the patient 102 and/or clinician 104 may operate the client interface 112 to initiate analysis of the sample 106 by the sample analyzer 108 and provision of analysis results (e.g., detected markers and/or marker levels) from the sample analyzer 108 to the risk estimation facility 116. Results of analysis of the results (received from the sample analyzer 108 or from the client interface 112) by the risk estimation facility 116 may be output to the client interface 112, such as by being received at the client interface 112 and displayed on the client computing device 110. In some embodiments, as mentioned above, the client interface 112 may include a web interface, such as one or more web pages into which values may be output and which may display results of the analysis by the risk estimation facility 116, but embodiments are not so limited. The client interface 112 may accept input in a variety of different formats, such as through speech recognition, text input, or other means, as embodiments are not limited in this respect.

The system 100 can include a server computing device 114, which may include a risk estimation facility 116 configured to analyze factors (e.g., derived from the sample 106, such as by the sample analyzer 108) for the patient 102 with a multiple myeloma (MM) precursor disease to determine a risk that the MM precursor disease of the patient 102 will progress into MM. These factors may include age of patient 102 and may include one or more time-varying markers, such as time-varying markers represented in a numeric form or as change indicators, such as trajectory indications. In some embodiments, the factors may include presence of and/or absence of certain genetic markers or mutations of interest, which may include 17 deletion, 17p deletion, 13 deletion, 13q deletion, or 1q gain. In some embodiments, the risk estimation facility 116 may receive information on the factors from the sample analyzer 108 and/or from the client interface 112. In some embodiments, the risk estimation facility 116 may output a risk, which may be one or more numeric values, that a condition of the patient 102 will progress (e.g., to a MM precursor disease or to MM) over one or more timeframes.

The system 100 can include a network 118 to facilitate communications among the sample analyzer 108, the client computing device 110, and the server computing device 114. The network 118 can be or include any one or more wired and/or wireless, local-and/or wide-area network, including one or more enterprise networks and/or the Internet.

While the example of FIG. 10 includes the client interface 112 on a client computing device 110 separate from the sample analyzer 108, it should be appreciated that embodiments are not so limited. In other embodiments, the client interface 112 may be an interface of the sample analyzer 108 and may be operated by the patient 102 and/or the clinician 104. Additionally or alternatively, while the risk estimation facility 116 is illustrated on a different computing device from the client computing device 110 and the sample analyzer 108, embodiments are not so limited. In other embodiments, the risk estimation facility may be implemented on the client computing device or the sample analyzer 108. In some embodiments, the client interface 112 may not be separate from the risk estimation facility 116, but instead may be implemented as a single program or software application. In some embodiments, a sample analyzer 108 may include the client interface 112 and the risk estimation facility 116, and the interface 112 and risk estimation facility 116 may be implemented within the same program or application executed on the sample analyzer 108.

FIG. 11 is a flowchart of a process 1100 that may be implemented in some embodiments to evaluate a risk of a patient for MM disease progression. Process 1100 can be implemented in some embodiments by the risk estimation facility, which can assess a risk that a condition of the patient will progress, such as into an MM precursor disease (e.g., MGUS or SMM) and/or into MM. By identifying individual patients with precursor myeloma diseases (MGUS and SMM) who are at the highest risks of disease progression to overt myeloma, treatment can intercept disease early and improve prognosis.

Prior to the start of process 1100, one or more models may have been trained that associate markers and/or values for markers with risks of MM precursor disease progression over one or more timeframes. Examples of training techniques are described in the Examples herein. A risk estimation facility may be configured with one or more of the models for use in determining a risk of MM disease progression, as discussed above and as discussed below in connection with process 1100.

Process 1100 begins in block 1102, in which a clinician obtains and/or a sample analyzer analyzes a sample of material from a patient to obtain information regarding one or more markers, which may include one or more marker values, information on presence or absence of markers, or other information regarding markers. For example, marker values can be obtained from a sample by sequencing, probes, immunoassay, biochip, protein biochip, nucleic acid biochip, or mass spectrometry, or using other techniques. Marker values may include information representing levels of a time-varying marker of a patient. In some embodiments, these values include a set of values that may include numeric values for one or more time-varying markers and/or change indicators such as trajectory values. A trajectory value may describe a change over time of a corresponding time-varying marker, such as whether a value is increasing or decreasing, or increasing, decreasing, or holding steady.

In some embodiments, block 1102 may include the risk estimation facility receiving (e.g., via a user interface, via a network communication, or otherwise) a multiple myeloma precursor disease progression risk request for a patient that includes information on one or more markers, such as one or more numeric values (e.g., for one or more time-varying marker) representing marker measurements associated with the patient. For example, a clinician can submit the multiple myeloma precursor disease progression request via a client user interface (e.g., client interface 112 of FIG. 10). In another example, the continuous variable can represent marker values described above. In another example, the marker measurement can be a measurement obtained by a clinician from analysis of a sample from the patient by using a sample analyzer (e.g., analyzer 108 of FIG. 10 operating on sample 106 from patient 102).

In some embodiments, a multiple myeloma precursor disease progression request can include numeric values for one, two, or more markers such as free light chain (FLC) ratio, M-spike concentration, age, creatinine concentration, and hemoglobin concentration, or any combination of these markers. In some embodiments, the multiple myeloma precursor disease progression request can include a numeric value for bone marrow plasma cell percent (BMPC %). In some embodiments, rather than include a numeric value for a marker, a change indicator may be received for a marker, which may indicate information regarding a change of the marker over time. Such a change indicator may indicate whether a change has occurred (e.g., within a time frame) and/or describe the change, such as by describing a rate of change or indicating whether a value has been increasing over time, decreasing over time, or holding steady. A change indicator may be quantitative or qualitative, such as by being a numeric indication or a categorical indication. A risk estimation facility may compare levels detected over time in blood or other material of the patient of a time-varying marker and determine a trajectory value describing the change over time of the marker.

In some embodiments, the multiple myeloma precursor disease progression request can include genetic markers, which may include information on a presence or absence of each of one or more genetic markers. Accordingly, in some embodiments, qualitative marker information may include 17 deletion, 17p deletion, 13 deletion, 13q deletion, and 1q gain.

In some embodiments, to assess the risk that the MM precursor disease of the patient will progress into MM, a risk estimation facility may determine whether an input value (e.g., continuous variable) resulting from a bone marrow biopsy has been received.

In some embodiments, the risk estimation facility may determine whether the multiple myeloma precursor disease progression request does not include information on one or more markers. In some such embodiments, the risk estimation facility may analyze the provided information, without the missing value. In other embodiments, rather than proceed without the information, the risk estimation facility may determine a value to be used in the analysis of values. In some such cases, to determine the value to be used in the analysis using the trained model, the risk estimation facility may determine the value to be a configured value, such as a default value. In other cases, to determine the value to be used in the analysis using the trained model, the risk estimation facility may analyze values that were received, or perform a computation using such provided values. For example, the risk estimation facility may determine a missing continuous variable prediction machine learning model from a set of missing continuous variable prediction machine learning models based on the missing continuous variable. Each missing continuous variable prediction machine learning model may include trained missing continuous variable prediction parameters that are trained for a particular missing continuous variable based on missing continuous variable prediction training data. In some such embodiments, the missing continuous variable prediction training data may include a continuous variable value of a known continuous variable paired with a known missing continuous variable value of the particular missing continuous variable. The risk estimation facility may utilize a missing continuous variable prediction machine learning model together with the values that were received for other markers to generate a probability distribution for the missing value, then select (e.g., randomly) a value from the probability distribution.

Block 1104 of the process 1100 includes selecting one or more models for analyzing received information regarding markers to determine MM disease progression risk. As discussed above, in some embodiments, a risk estimation facility may be configured with multiple models, such as models that use different markers to predict risk. For example, one model may account for BMPC % while another does not, or one model may account for genetic markers while another does not. One or more of the models may be Precursor Asymptomatic Neoplasms by Group Effort Analysis (PANGEA) models, examples of which are discussed herein. In some embodiments, to select a model, the risk estimation facility may determine what information was received in the risk request, or otherwise determine the marker(s) for which information was received. For example, depending on whether any trajectory information is received (e.g., hemoglobin trajectory information), whether any information derived from a bone marrow biopsy is received (e.g., BMPC %), and/or whether any genetic data is received, the risk estimation facility may select between models and choose the one that is operable with the information that was received. As mentioned above, in some cases the risk estimation facility may determine a value for a marker when no value was received for that marker. In some embodiments, the risk estimation facility may select a model by reviewing the marker(s) for which information is available, both received information or determined/generated information. In other embodiments, the risk estimation facility may determine/generate a value for a marker after a model is selected, and generate information for those markers for which the selected model calls for an input value and where a value was not otherwise received or input.

In some embodiments, a PANGEA machine learning model includes trained PANGEA parameters that are trained for a particular combination of continuous variables based on training data. For example, the PANGEA Model can leverage machine learning (ML) techniques in the MM setting. In another example, the PANGEA model accounts for genomic and circulating tumor cell data. In some embodiments, the training data includes historical marker measurements for the particular combination of continuous variables paired with known historical trajectories of the particular combination of continuous variables. For example, the PANGEA model can be developed based upon a cohort of patients with longitudinal data including MGUS and SMM at baseline. In some embodiments, the PANGEA machine learning model includes PANGEA parameters trained for a particular combination of continuous and categorical variables based on the training data. For example, the PANGEA machine learning model can include time-varying clinical markers without arbitrary cutoffs to model how precursor progression risk to MM evolves for a patient over time, regardless of whether a bone marrow biopsy (BMbx) is conducted. In another example, the model can be a multivariate Cox model with time-varying and trajectory markers with or without bone marrow data to estimate progression risk.

In block 1106, the risk estimation facility analyzes marker values with the trained model (e.g., PANGEA machine learning model). In some embodiments, the risk estimation facility analyzes the levels of time-varying markers determined from material (e.g., blood, tissue) of the patient. In some embodiments, the risk estimation facility analyzes values representing a detected level of a time-varying marker found in the blood or other material of a sample of the patient. For example, the risk estimation facility can use time-varying markers (e.g., monoclonal protein, free light chain, age, creatinine, bone marrow plasma cell percentage) and dynamic trajectories (e.g., hemoglobin) to predict progression from precursor disease (e.g., MGUS or SMM) to MM.

In some embodiments, the risk estimation facility can analyze, with the trained model, an age of the patient. In some embodiments, the analysis can include analyzing the age of the patient together with values. In some embodiments, the risk estimation facility can ingest a numeric variable for one or more time-varying markers, age, and/or one or more categorical/qualitative values to produce the predicted risk throughout one or more prediction periods. A numeric value may be a value of a measurement of a marker from a sample of the patient, or may be a numeric value indicating a change in a value of a measurement such as a rate of change. A numeric value for a marker may be a continuous value. Such a continuous value may be a real number in some cases, or may be a rational or non-integer number. In some embodiments, a continuous value may be stored and used to a configured number of significant digits, such as two, three, four, or five significant digits. A continuous value may be differentiated from a discrete value in some embodiments because the discrete value may be able to take only a value from a known set or group of values, whereas the continuous value may take any value within a defined range and then be represented in accordance with the configured precision. Categorical/qualitative values may include values indicating presence or absence of markers. Such categorical/qualitative values may also include a change indicator indicating whether a value of a marker has changed or not, or whether the value of the marker is increasing or decreasing, or increasing, decreasing, or holding steady. This analysis can include, in some embodiments, the risk estimation facility analyzing the age of the patient and the values using a trained model that is trained to evaluate risk of MM precursor disease progressing into MM.

As discussed above, the risk estimation facility selects the model(s) in block 1104 and then analyzes values with the selected model(s) in block 1106. Accordingly, how the analysis of block 1106 proceeds may vary between patients and/or between embodiments.

In some cases, to analyze the age of the patient with the trained model together with values representing levels of time-varying markers, the risk estimation facility can analyze, with the trained model, a numeric age of the patient, a numeric FLC ratio for the patient, a numeric value for M-spike for the patient, a numeric value for creatinine for the patient, and a trajectory value indicating whether an amount of hemoglobin for the patient has been increasing or decreasing.

In some cases, to analyze using a model, the risk estimation facility may, in response to determining that an input value resulting from a bone marrow biopsy has been received, analyze, using a first trained model that accounts for biopsy values, the age of the patient together with the values and the input value for the biopsy, which may be a BMPC % for the patient. In some embodiments, in response to determining that an input value resulting from a bone marrow biopsy has not been received, the risk estimation facility can analyze the age of the patient together with the values using a second trained model different from the first trained model.

In some cases, to analyze, using a model, the age and the values representing a level of a time-varying marker, the risk estimation facility can analyze the age, the values, and an indicator of whether the patient has been detected to have a genetic marker (e.g., disease-related marker). In some embodiments, to analyze the age, the values, and the indicator of whether the patient has been detected to have the genetic marker, the risk estimation facility can analyze the age, the values, and the indicator that indicates whether a patient has been found to have a genetic marker for a group of genetic markers consisting of 17 deletion, 17p deletion, 13 deletion, 13q deletion, and 1q gain. In some embodiments, to analyze the age, the values, and the indicator of whether the patient has been detected to have the genetic marker, the risk estimation facility can analyze the age, the plurality of values, and indicators that indicate whether a patient has been found to have a corresponding one of 17 deletion, 17p deletion, 13 deletion, 13q deletion, and/or 1q gain. For example, the risk estimation facility can account for increased progression of risk observed in the patient with 1q gain, 13/13q deletion, or 17/17p deletion, and/or MYC alterations.

In some embodiments, the risk estimation facility can analyze, with the trained model, a FLC ratio for the patient. In some embodiments, the risk estimation facility can analyze, with the trained model, a level of M-spike for the patient. In some embodiments, the risk estimation facility can analyze, with the trained model, a level of creatinine for the patient. For example, since creatinine is related to kidney function and MM is a blood disease, the creatinine level can be indicative of the risk that the MM precursor disease of the patient will progress into MM. In some embodiments, the risk estimation facility can analyze, with the model, a trajectory value indicating whether an amount of hemoglobin increased or decreased.

In block 1108, the risk estimation facility generates one or more risk predictions for one or more time frames. In some embodiments, as a result of the analysis with the selected model(s), the risk estimation facility can generate a value indicating the risk that the MM precursor disease of the patient will progress into MM. For example, the risk estimation facility can be used to compute individual multiple myeloma (MM) progression probabilities for the patient that has precursor conditions.

In some embodiments, the risk estimation facility can utilize the model(s) to ingest the continuous variables and produce a predicted risk throughout a set of prediction periods that represent a future risk of progression for the patient based on the trained PANGEA parameters. In some embodiments, to assess the risk that the MM precursor disease of the patient will progress into MM, the risk estimation facility can assess the risk that the MM precursor disease of the patient will, within a timeframe, progress into MM. In some embodiments, to assess the risk that the MM precursor disease of the patient will progress into MM within the timeframe, the risk estimation facility can assess a risk for each timeframe that the MM precursor disease will, within the corresponding timeframe, progress into MM. In some embodiments, to generate the value indicating the risk, the risk estimation facility can generate a numeric value indicating the risk.

Block 1110 of the process 1100 includes outputting risk predictions for presentation. In some embodiments, the risk estimation facility can output the value indicating that risk for the patient. In some embodiments, to output the value indicating the risk, the risk estimation facility can output a numeric value. For example, the risk estimation facility can use the risk prediction to stratify patients based on their individual predicted risk. This allows for clustering of the patient at higher risk of disease progression and selection of patient populations for the development of precursor clinical trials.

In some embodiments, the risk estimation facility can transmit a multiple myeloma precursor disease progression response. For example, the risk estimation facility can transmit to a client computing device (e.g., client computing device 110 of FIG. 10). The multiple myeloma precursor disease progression response can include the predicted risk throughout the prediction period. In some embodiments, the multiple myeloma precursor disease progression response can include the probability distribution of potential missing continuous variable values.

In some embodiments, the risk estimation facility can render a graphical plot depicting the future risk of progression based on the continuous variable and the categorical variable. The risk estimation facility can configure the multiple myeloma precursor disease progression response to cause the client computing device to render a graphical plot depicting the future risk of progression based on the continuous variables. For example, the client computing device can render the graphical plot for display in the client interface to the patient or the clinician. The risk estimation facility can configure the multiple myeloma precursor disease progression response to cause the computing device to render a graphical plot that depicts the probability distribution of potential missing continuous variable values. For example, the clinician can use the graphical plot to decide the appropriate time intervals for a follow-up for the patient.

Once the risk predictions are output in block 1110, the process 1100 ends and may be repeated as desired.

Techniques operating according to the principles described herein may be implemented in any suitable manner. Included in the discussion above are a series of flow charts showing the steps and acts of various processes for estimation of a patient's risk of MM precursor disease progression. The processing and decision blocks of the flow charts above represent steps and acts that may be included in algorithms that carry out these various processes. Algorithms derived from these processes may be implemented as software integrated with and directing the operation of one or more single-or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionalities may be implemented in a single functional facility. It should also be appreciated that, in some implementations, some of the functional facilities described herein may be implemented together with or separately from others (i.e., as a single unit or separate units), or some of these functional facilities may not be implemented.

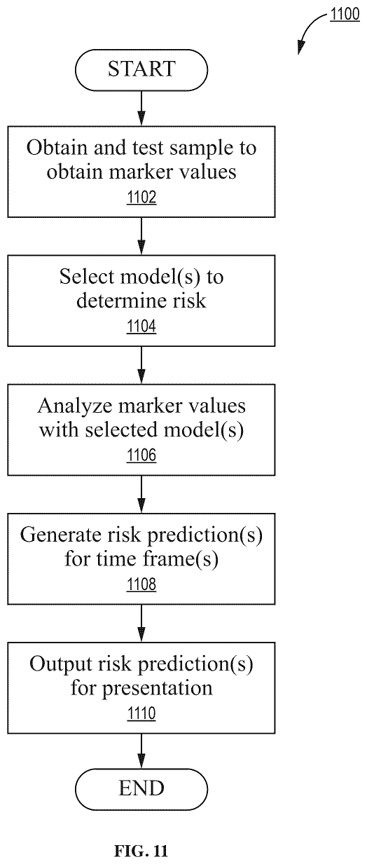

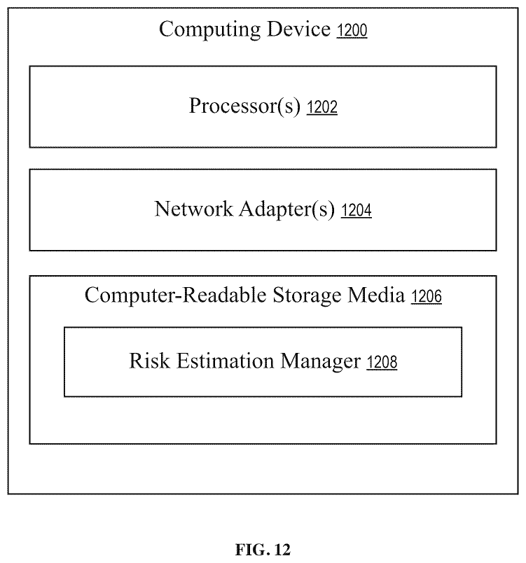

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 1206 of FIG. 12 described below (i.e., as a portion of a computing device 1200) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 10, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing devices sharing processing power and jointly carrying out the techniques described herein, a single computing device or coordinated system of computing devices (co-located or geographically distributed) dedicated to executing the techniques described herein, one or more Field-Programmable Gate Arrays (FPGAs) for carrying out the techniques described herein, or any other suitable system.

FIG. 12 illustrates one exemplary implementation of a computing device in the form of a computing device 1200 that may be used in a system implementing techniques described herein, although others are possible. It should be appreciated that FIG. 12 is intended neither to be a depiction of necessary components for a computing device to execute a risk estimation facility in accordance with the principles described herein, nor a comprehensive depiction.

Computing device 1200 may comprise at least one processor 1202, a network adapter 1204, and computer-readable storage media 1206. Computing device 1200 may be, for example, a desktop or laptop personal computer, a personal digital assistant (PDA), a smart mobile phone, a server, a wireless access point or other networking element, or any other suitable computing device. Network adapter 1204 may be any suitable hardware and/or software to enable the computing device 1200 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 1206 may be adapted to store data to be processed and/or instructions to be executed by processor 1202. Processor 1002 enables processing of data and execution of instructions. The data and instructions may be stored on the computer-readable storage media 1206.

The data and instructions stored on computer-readable storage media 1206 may comprise computer-executable instructions implementing techniques which operate according to the principles described herein. In the example of FIG. 12, computer-readable storage media 1206 stores computer-executable instructions implementing various facilities and storing various information as described above. Computer-readable storage media 1206 may store risk estimation manager 1208.

While not illustrated in FIG. 12, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

### Detection of Markers

The present disclosure provides for the detection of a variety of clinical variables associated with MM and/or an MM precursor disease (e.g., MGUS, SMM) for use in the claimed risk assessment methods. In some embodiments, the clinical variable is the level of a marker, including but not limited to creatinine, hemoglobin, serum M-protein, serum free light chain (FLC) ratio, bone marrow plasma cell percent (BMPC %), total protein, IgA, IgM, IgG, kappa free light chain (FLC), lambda FLC, calcium, albumin, LDH, beta-2 microglobulin, M-spike, LDH, beta-2 microglobulin, and urine M-protein. In some embodiments, a level of marker is detected at a single time point or serial values are annotated over time (e.g., days, weeks, months). In some embodiments, levels of a marker is detected at 3, 4, 5, 6, 7, 8, 9, 10, 11, or 12-month time intervals from the date of MGUS or SMM diagnosis. In some embodiments, levels of a marker described herein are detected prior to, during or following initiation of precursor treatment. In some embodiments, levels of a marker are detected in a biological sample (e.g., blood, serum, plasma, or bone marrow biopsy).

The markers of this disclosure can be detected by any suitable method. The methods described herein can be used individually or in combination for a more accurate detection of the markers (e.g., biochip in combination with mass spectrometry, immunoassay in combination with mass spectrometry, single cell RNA sequencing, and the like).

One of skill in the art is familiar with how to detect levels of markers in a sample. For example, commercial kits and/or well developed methods are available for detection of creatinine (e.g., the “Creatinine Assay Kit” from Cell Biolabs, Inc.), free light chain ratio (see, e.g., Tosi, et al. Ther Adv Hematol 4:37-41 (2013)), M-spike (see, e.g., Noori, et al. Clinical Chemistry and Laboratory Medicine 59:1963-1971 (2021)), or hemoglobin (e.g., the “Hemoglobin Assay Kit” available from Millipore Sigma) in a sample.

Detection paradigms that can be employed in the methods described herein include, but are not limited to, optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are described below.

Detection By Sequencing and/or Probes

In particular embodiments, the markers of the disclosure are analyzed by a sequencing- and/or probe-based technique (e.g., RNA-seq).

RNA sequencing (RNA-Seq) is a powerful tool for transcriptome profiling. In embodiments, to mitigate sequence-dependent bias resulting from amplification complications to allow truly digital RNA-Seq, a set of barcode sequences can be used to ensure that every cDNA molecule prepared from an mRNA sample is uniquely labeled by random attachment of barcode sequences to both ends (see, e.g., Shiroguchi K, et al. Proc Natl Acad Sci USA. 2012 Jan. 24;109 (4): 1347-52). After PCR, paired-end deep sequencing can be applied to read the two barcodes and cDNA sequences. Rather than counting the number of reads, RNA abundance can be measured based on the number of unique barcode sequences observed for a given cDNA sequence. The barcodes may be optimized to be unambiguously identifiable. This method is a representative example of how to quantify a whole transcriptome from a sample.

Detecting a target polynucleotide sequence or fragment thereof associated with a marker that hybridizes to a probe sequence may involve sequencing, FACS, qPCR, RT-PCR, a genotyping array, and/or a NanoString assay (see, e.g., Malkov, et al. “Multiplexed measurements of gene signatures in different analytes using the Nanostring nCounter™ Assay System”, BMC Research Notes, 2: Article No: 80 (2009)), or any of various other techniques known to one of skill in the art. Various detection methods may be used and are described as follows.

Preparation of a library for sequencing may involve an amplification step. Amplification may involve thermocycling or isothermal amplification (such as through the methods RPA or LAMP). Cross-linking may involve overlap-extension PCR or use of ligase to associate multiple amplification products with each other. Amplification can refer to any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a marker.

Detection of the expression level of a marker can be conducted in real time in an amplification assay (e.g., qPCR). In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dyes suitable for this application include, as non-limiting examples, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

Other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are taught, for example, in U.S. Pat. No. 5,210,015.

Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference).

The sequencing of a polynucleotide can be carried out using any suitable commercially available sequencing technology. In embodiments, the sequencing of a polynucleotide is carried out using a chain termination method of DNA sequencing (e.g., Sanger sequencing). In some embodiments, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLID sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

In embodiments, levels of markers in a sample are quantified using targeted sequencing. Methods for targeted sequencing are well known in the art (see, e.g., Rehm, “Disease-targeted sequencing: a cornerstone in the clinic”, Nature Reviews Genetics, 14:295-300 (2013)).

In embodiments, a probe comprises a molecular identifier, such as a fluorescent or chemiluminescent label, a radioactive isotope label, an enzymatic ligand, or the like. The molecular identifier can be a fluorescent label or an enzyme tag, such as digoxigenin, β-galactosidase, urease, alkaline phosphatase or peroxidase, avidin/biotin complex.

Methods used to detect or quantify binding of a probe to a target marker will typically depend upon the molecular identifier. For example, radiolabels may be detected using photographic film or a phosphoimager. Fluorescent markers may be detected and quantified using a photodetector to detect emitted light. Enzymatic labels can be detected by providing the enzyme with a substrate and measuring the reaction product produced by the action of the enzyme on the substrate; and colorimetric labels can be detected by visualizing a colored label.

Specific non-limiting examples of molecular identifiers include radioisotopes, such as 32P, 14C, 125I, 3H, and 131I, fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a molecular identifier, streptavidin bound to an enzyme (e.g., peroxidase) may further be added to facilitate detection of the biotin.

Examples of fluorescent molecular identifiers include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

A fluorescent molecular identifier may be a fluorescent protein, such as blue fluorescent protein, cyan fluorescent protein, green fluorescent protein, red fluorescent protein, yellow fluorescent protein or any photoconvertible protein. Colorimetric molecular identifiers, bioluminescent molecular identifiers and/or chemiluminescent molecular identifiers may be used in embodiments of the disclosure.

Detection of a molecular identifier may involve detecting energy transfer between molecules in a hybridization complex by perturbation analysis, quenching, or electron transport between donor and acceptor molecules, the latter of which may be facilitated by double stranded match hybridization complexes. The fluorescent molecular identifier may be a perylene or a terrylen. In the alternative, the fluorescent molecular identifier may be a fluorescent barcode.

The molecular identifier may be light sensitive, wherein the label is light-activated and/or light cleaves the one or more linkers to release the molecular cargo. The light-activated molecular cargo may be a major light-harvesting complex (LHCII). In another embodiment, the fluorescent molecular label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

In embodiments, the molecular identifier is a microparticle, including, as non-limiting examples, quantum dots (Empodocles, et al., Nature 399:126-130, 1999), or gold nanoparticles (Reichert et al., Anal. Chem. 72:6025-6029, 2000).

### Detection by Immunoassay

In particular embodiments, the markers of the disclosure are measured by immunoassay. Immunoassay typically utilizes an antibody (or other agent that specifically binds the marker) to detect the presence or level of a marker in a sample. Antibodies can be produced by methods well known in the art, e.g., by immunizing animals with the markers. Markers can be isolated from samples based on their binding characteristics. Alternatively, if the amino acid sequence of a polypeptide marker is known, the polypeptide can be synthesized and used to generate antibodies by methods well known in the art.

This disclosure contemplates traditional immunoassays including, for example, Western blot, sandwich immunoassays including ELISA and other enzyme immunoassays, fluorescence-based immunoassays, and chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the marker is attached to the surface of an MS probe, such as a pre-activated ProteinChip array. The marker is then specifically captured on the biochip through this reagent, and the captured marker is detected by mass spectrometry.

### Detection by Biochip

In embodiments, a sample is analyzed by means of a biochip (also known as a microarray). The polypeptides and nucleic acid molecules of the disclosure are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In embodiments, a sample is analyzed by means of a protein biochip (also known as a protein microarray). Such biochips are useful in high-throughput low-cost screens to identify alterations in the expression or post-translation modification of a marker, or a fragment thereof. In embodiments, a protein biochip of the disclosure binds a marker present in a sample and detects an alteration in the level of the marker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the disclosure) are spotted on a substrate using any convenient method known to the skilled artisan (e.g., by hand or by inkjet printer).

In embodiments, the protein biochip is hybridized with a detectable probe. Such probes can be polypeptides, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); tissue (e.g., bone marrow), a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, CA), Zyomyx (Hayward, CA), Packard BioScience Company (Meriden, CT), Phylos (Lexington, MA), Invitrogen (Carlsbad, CA), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the disclosure, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application W095/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g., RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed as defined above.

The removal of nonhybridized probes may be accomplished, for example, by washing. The washing steps that follow hybridization can also vary in stringency, as defined above.

Detection systems for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In embodiments, the markers of this disclosure are detected by mass spectrometry (MS). Mass spectrometry is a well-known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2):880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 2005/0023454; 2005/0035286; U.S. Pat. No. 5,800,979 and the references disclosed therein.

### Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

### Matrix-Assisted Laser Desorption/Ionization (MALDI) and Electrospray Ionization (ESI)

In embodiments, the mass spectrometric technique for use in the disclosure is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the disclosure to produce an array of spots on a collecting membrane, and the array may be analyzed. Subsequently, automated output of the results is provided by using a server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MSⁿ analysis to be performed. Tandem mass spectrometry allows multiple reactions to be monitored at the same time.

Capillary infusion may be employed to introduce the marker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high-performance liquid chromatography (HPLC) to a mass spectrometer for integrated sample separation/and mass spectrometer analysis.

Quadrupole mass analyzers may also be employed as needed to practice the disclosure. Fourier-transform ion cyclotron resonance (FTMS) can also be used for some disclosure embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in the disclosure is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the markers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

A biospecific adsorbent is an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen comprise surfaces having chromatographic or biospecific adsorbents attached thereto at addressable locations. Ciphergen's ProteinChip® arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidazole, epoxide) and PG-20 (protein G coupled through acyl-imidazole). Hydrophobic ProteinChip arrays have isopropyl or nonylphenoxy-poly (ethylene glycol) methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidazole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Application Publication No. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. 2003/0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the marker or markers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound markers.

In yet another method, one can capture the markers with a solid-phase bound immuno-adsorbent that has antibodies that bind the markers. After washing the adsorbent to remove unbound material, the markers are eluted from the solid phase and detected by applying to a SELDI biochip that binds the markers and analyzing by SELDI.

The markers bound to the substrates are detected in a gas phase ion spectrometer such as a time-of-flight mass spectrometer. The markers are ionized by an ionization source such as a laser, the generated ions are collected by an ion optic assembly, and then a mass analyzer disperses and analyzes the passing ions. The detector then translates information of the detected ions into mass-to-charge ratios. Detection of a marker typically will involve detection of signal intensity. Thus, both the quantity and mass of the marker can be determined.

### Treatments

Methods of inhibiting and/or treating cancer and tumors (e.g., a multiple myeloma) in a subject with cancer or a predisposition for developing cancer as identified by methods of the disclosure are also contemplated. Methods described herein are useful as clinical or companion diagnostics for therapies or can be used to guide treatment decisions based on clinical response/resistance.

Frontline therapy for MM includes either conventional chemotherapy or high-dose chemotherapy (HDT) supported by autologous or allogeneic stem cell transplantation (SCT), depending on patient characteristics such as performance status, age, availability of a sibling donor, comorbidities, and, in some cases, patient and physician preferences. Other treatments include: bortezomib, thalidomide, lenalidomide, dexamethasone, cyclophosphamide, melphalan, and stem cell transplant. For a patient under 70 years of age, autologous stem cell transplant is proposed after induction.

Non-limiting examples of agents suitable for use to treat a multiple myeloma include a chemotherapeutic agent, radiation, or immunotherapy. Any suitable therapeutic treatment for a particular cancer may be administered. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, tamoxifen, taxol, topotecan hydrochloride, trastuzumab, vinblastine, vincristine and vinorelbine tartrate. Further non-limiting examples of chemotherapeutic agents include an alkylating agent (e.g. busulfan, chlorambucil, cisplatin, cyclophosphamide (Cytoxan), dacarbazine, ifosfamide, mechlorethamine (mustargen), and melphalan), a topoisomerase inhibitor, an antimetabolite (e.g. 5-fluorouracil (5-FU), cytarabine (Ara-C), fludarabine, gemcitabine, and methotrexate), an anthracycline, an antitumor antibiotic (e.g. bleomycin, dactinomycin, daunorubicin, doxorubicin (Adriamycin), and idarubicin), an epipodophyllotoxin, nitrosureas (e.g. carmustine and lomustine), topotecan, irinotecan, doxorubicin, etoposide, mitoxantrone, bleomycin, busultan, mitomycin C, cisplatin, carboplatin, oxaliplatin and docetaxel.

In embodiments, response to therapy is measured using the methods provided herein (e.g., through molecular characterization of circulating multiple myeloma cells). In embodiments, response to therapy is measured by a reduction in M protein levels in serum and/or urine and the reduction in size or disappearance of plasmacytomas. The international uniform response criteria for MM have expanded upon the European Group for Blood and Marrow Transplantation criteria to provide a more comprehensive evaluation system (Durie B. G. et al., Leukemia, 20:1467-73 (2006)). Importantly, achievement of response has been associated with improved survival in SCT trials with high-dose therapy. Similarly, time to progression (TTP) has been shown to be an important surrogate for improved survival. Despite high response rates to frontline therapy, virtually all patients eventually relapse. Table B shows the international uniform response criteria for MM.

TABLE B
International uniform response criteria for multiple myeloma (MM)

| Response | |
|---|---|
| Subcategory | Response Criteria |
| CR (complete | Negative immunofixation on the serum |
| response) | and urine and disappearance of any |
| | soft tissue plasmacytomas and ≤5% |
| | plasma cells in bone marrow |
| sCR (stringent | CR as described above, plus: |
| complete | normal free light chain (FLC) ratio |
| response) | and absence of clonal cells in bone |
| | marrow by immunohistochemistry |
| | or immunofluorescence |
| VGPR (very good | Serum and urine M-protein detectable |
| partial response) | by immunofluorescence but not on |
| | electrophoresis or 90% or greater |
| | reduction in serum M-protein plus urine |
| | M-protein level <100 mg per 24 hours |
| PR (partial | ≥50% reduction of serum M-protein |
| response) | and reduction in 24-h urinary M-protein |
| | by ≥90% or to <200 mg per 24 h |
| | If the serum and urine M-protein are |
| | unmeasurable, a ≥50% decrease in the |
| | difference between involved and |
| | uninvolved FLC levels is required in place |
| | of the M-protein criteria |
| | If serum and urine M-protein are |
| | unmeasurable, and serum free light assay |
| | is also unmeasurable, ≥50% reduction |
| | in plasma cells is required in place of |
| | M-protein, provided baseline bone |
| | marrow plasma cell percentage was ≥30% |
| | In addition to the above listed criteria, |
| | if present at baseline, a ≥50% |
| | reduction in the size of soft tissue |
| | plasmacytomas is also required. |
| SD (stable | Not meeting criteria for CR, VGPR, |
| disease) | PR or progressive disease |

In embodiments, the subject has been diagnosed with cancer or is at risk of developing a multiple myeloma.

For therapeutic use, administration of an agent can begin at the detection or surgical removal of tumors. This can be followed by boosting doses until at least symptoms are substantially abated and for a period thereafter.

The pharmaceutical compositions for therapeutic treatment are intended for parenteral, topical, nasal, oral or local administration. Preferably, the pharmaceutical compositions are administered parenterally, e.g., intravenously, subcutaneously, intradermally, or intramuscularly. The disclosure provides compositions for parenteral administration which comprise a solution of a suitable agent dissolved or suspended in an acceptable carrier, preferably an aqueous carrier. A variety of aqueous carriers may be used, e.g., water, buffered water, saline, glycine, hyaluronic acid, and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents, and the like, for example, sodium acetate, sodium lactate, sodium chloride, potassium chloride, calcium chloride, sorbitan monolaurate, triethanolamine oleate, etc.

In an embodiment, the cancer therapeutic is an immunotherapeutic (e.g., an antibody). The cancer therapeutic can be a chimeric antigen receptor (CAR) T cell. The immunotherapeutic may be a cytokine therapeutic (such as an interferon or an interleukin), a dendritic cell therapeutic or an antibody therapeutic, such as a monoclonal antibody. In an embodiment, the immunotherapeutic is a neoantigen (see, e.g., U.S. Pat. No. 9,115,402 and US Patent Application Publication Nos. 20110293637, 20160008447, 20160101170, 20160331822 and 20160339090).

### Kits

The disclosure also provides kits for use in embodiments of the methods provided herein. Kits of the instant disclosure may include one or more containers comprising one or more agents suitable for use in detecting markers in a subject and/or for treatment of a multiple melanoma (MM). In some embodiments, the kits further include instructions for use in accordance with the methods of this disclosure. In some embodiments, these instructions comprise a description of use of the agent for use in detecting markers and/or use of the agent for treatment of a multiple myeloma (MM). The kit may further comprise a description of how to analyze and/or interpret data.

Instructions supplied in the kits of the instant disclosure can be written instructions on a label or package insert (e.g., a paper sheet included in the kit), or machine-readable instructions (e.g., instructions carried on a magnetic or optical storage disk). Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. For example, the kit comprises a container and a label or package insert(s) on or associated with the container.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Particularly useful techniques for particular embodiments will be discussed in the sections that follow.

Described below are examples of ways in which techniques described herein may be implemented. It should be appreciated that these examples are merely illustrative, that embodiments are not limited to operating in accordance with the specific examples shown in the figures and discussed below, and that other embodiments are possible.

EXAMPLES

### Example 1: The Precursor Asymptomatic Neoplasms by Group Effort Analysis (PANGEA) Project was Representative of Monoclonal Gammopathy of Undetermined Significance (MGUS) and Smoldering Multiple Myeloma (SMM) Patient Populations

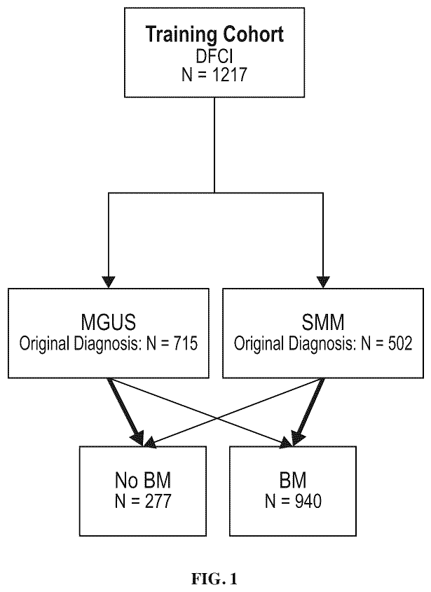

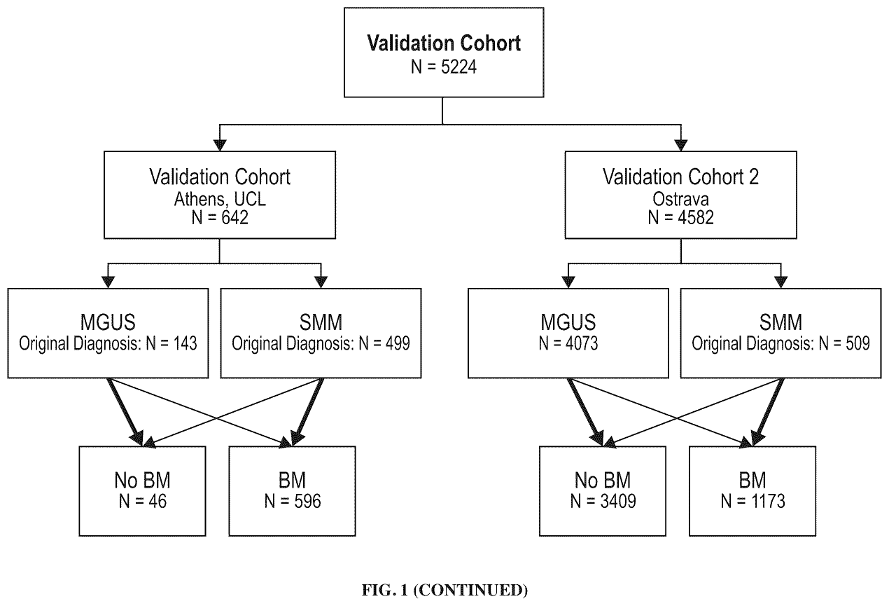

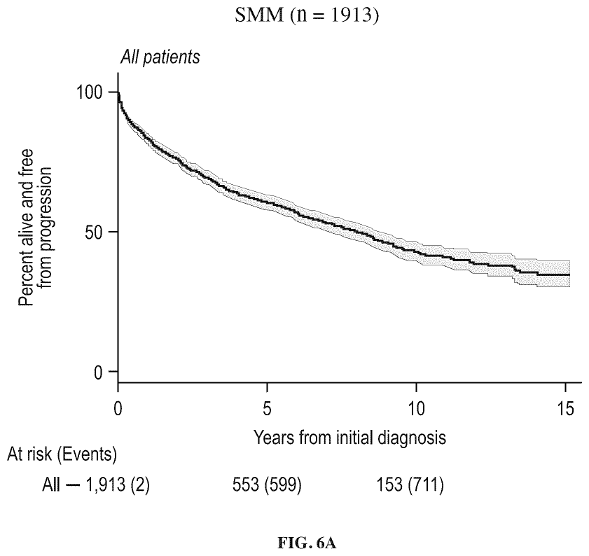

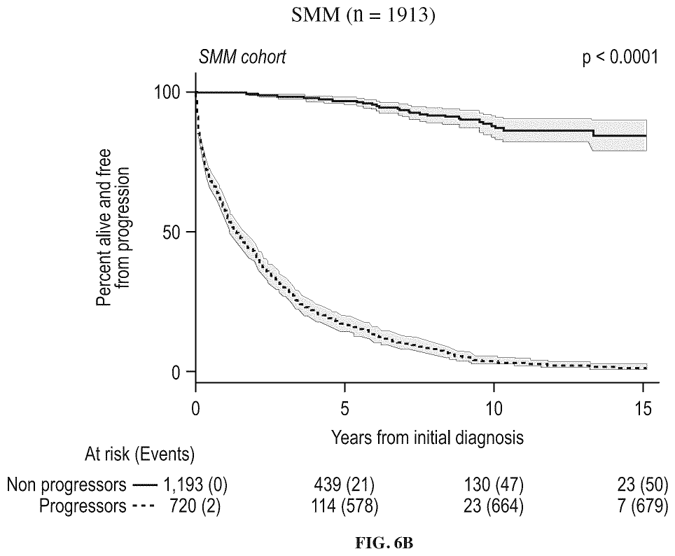

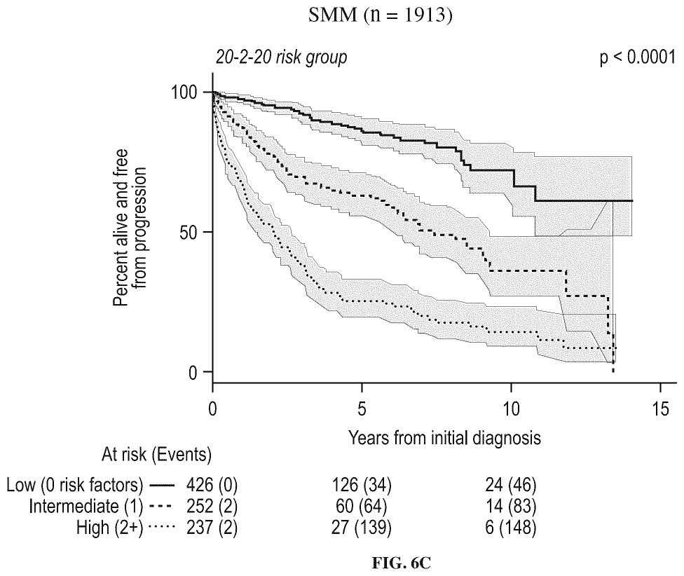

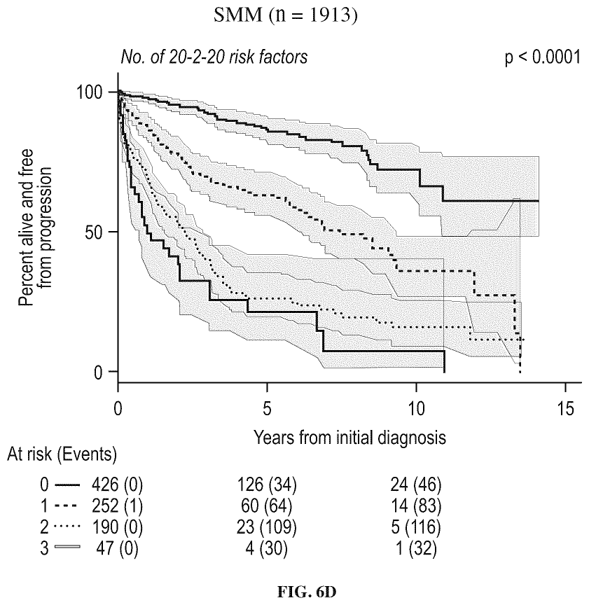

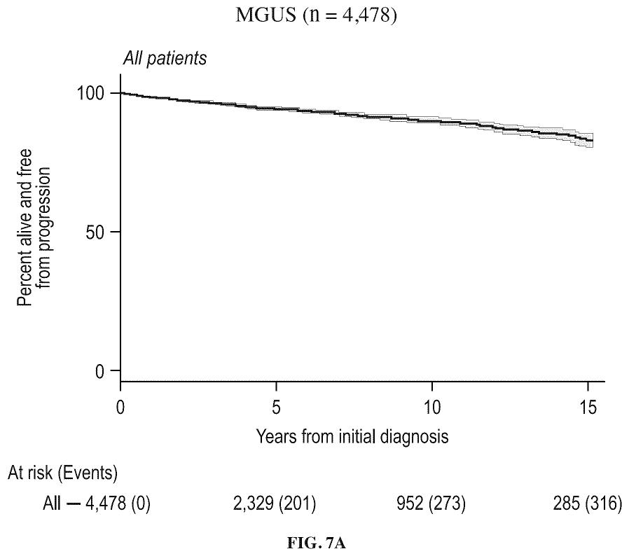

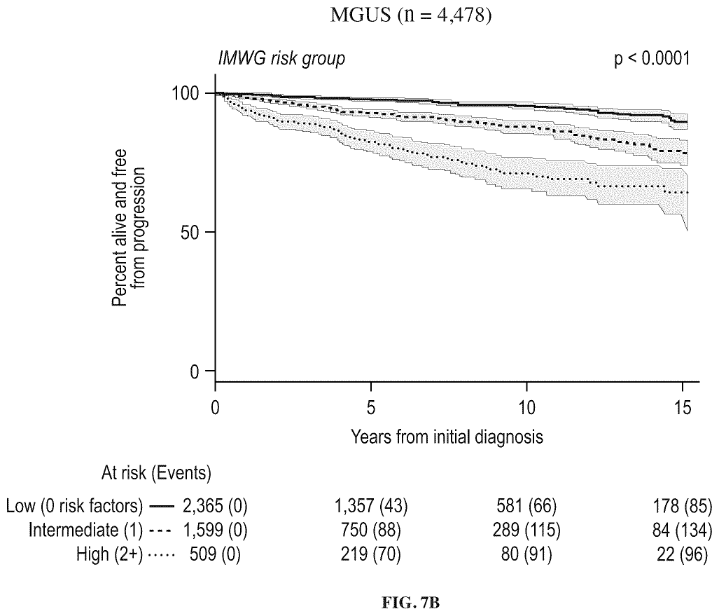

An experiment was undertaken to demonstrate that the PANGEA project was representative of MGUS and SMM patient populations. The PANGEA Project included SMM (FIGS. 6A-6D) and MGUS (FIGS. 7A and 7B) patients within three independent cohorts: a PANGEA Model was built from the Training Cohort and validated against Validation Cohort 1 and Validation Cohort 2. The distribution of markers within the Training Cohort was representative across 20/2/20 risk groups (Table 1). The average patient age at initial diagnosis for the Training Cohort, Validation Cohort 1, and Validation Cohort 2 was 62, 64, and 65.2 years, median number of laboratory timepoints of 8, 6, and 1, and median time of follow-up of 4.2, 2.9, and 3.6 years, respectively (FIG. 1 ). The differences between Validation Cohort 1 and 2 allowed for an evaluation of how the PANGEA Model performed in a precursor cohort with extensive longitudinal follow-up (Validation Cohort 1) and for precursor patients with limited data (Validation Cohort 2) as well as for a precursor cohort with similar progression rates (Validation Cohort 1; 2.23% progression to MM by visit 3) to the Training Model (2.18% progression to MM by Visit 3) and disparate progression rates (Validation Cohort 2; 0.54% progression to MM by Visit 3).

TABLE 1
Distribution of laboratory measurements across 20/2/20 risk
groups for the PANGEA Training Cohort. The term
“MGUS” represents “monoclonal gammopathy of undetermined
significance,” and the term “IQR” represents “interquartile range.”

| | 20/2/20 Risk Groups | | | Total |
|---|---|---|---|---|
| | | INTER- | | n = 12217 |
| MGUS | LOW | MEDIATE | HIGH | (%) |

Number of Bone Marrow Biopsies

| | | | | |
|---|---|---|---|---|
| Median (IQR) | 1 (0-2) | 1 (1-2) | 2 (1-3) | 2 (1-3) |
| 0 | 307 (43) | 6 (2) | 4 (3) | 5 (6) |
| | 214 (30) | 167 (58) | 55 (43) | 25 (30) |
| | 95 (13) | 56 (19) | 28 (22) | 30 (37) |
| 3 | 55 (8) | 36 (12) | 18 (14) | 12 (15) |
| 4 | 22 (3) | 18 (6) | 12 (9) | 8 (10) |
| 5 | 11 (2) | 6 (2) | 7 (5) | — |
| 6 | 7 (1) | — | 1 (1) | 1 (1) |
| 7 | 3 (0) | 1 (0) | 2 (2) | 1 (1) |
| 8 | 3 (0) | — | — | — |
| 9 | 1 (0) | — | 1 (1) | — |

Biopsy Conducted At Time of Diagnosis

| | | | | | |
|---|---|---|---|---|---|
| Yes (%) | 180 (29) | 253 (41) | 115 (19) | 70 (11) | 618 (51) |

Months Between Biopsies (averaged within patient)

| | | | | |
|---|---|---|---|---|
| Median (IQR) | 2.1(1.0-4.1) | 1.3 (1.0- | 1.0 (0.74- | 1.0 (0.46- |
| | | 2.9) | 1.5) | 2.0) |

Number of Laboratory Measurements

| | | | | |
|---|---|---|---|---|
| Median Creatinine | 5 (3-10) | 6 (3-11) | 5 (3-8) | 4 (2-9) |
| (IQR) | | | | |
| Median Hemoglobin | 5(3-10) | 6 (3-11) | 5 (3-9) | 4 (2-9) |
| (IQR) | | | | |
| Median Involved: | 5 (2-9) | 6 (3-11) | 5 (3-9) | 4 (2-9) |
| Uninvolved | | | | |
| FLC Ratio (IQR) | | | | |
| Median M-Spike | 4 (1-9) | 6 (3-10) | 4 (2-7) | 3 (2-6) |
| (IQR) | | | | |
| Median BMPC % | 1 (0-2) | 1 (1-2) | 2 (1-3) | 2 (1-3) |
| (IQR) | | | | |

### Example 2: Age, Creatinine, Hemoglobin, Free Light Chain (FLC) Ratio, M-Spike, and Bone Marrow Plasma Cell Percent (BMPC %) as Continuous Variables Predicted Precursor Disease Progression to Multiple Myeloma (MM)

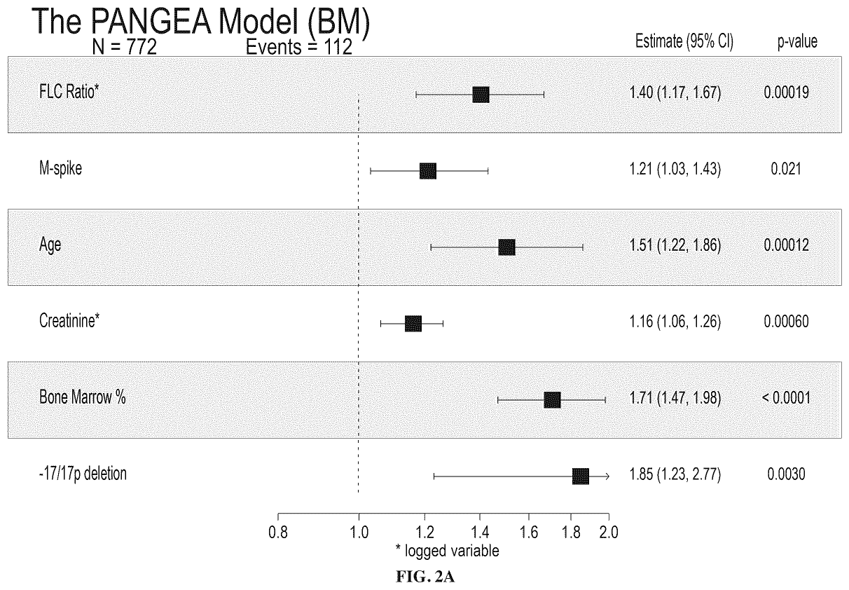

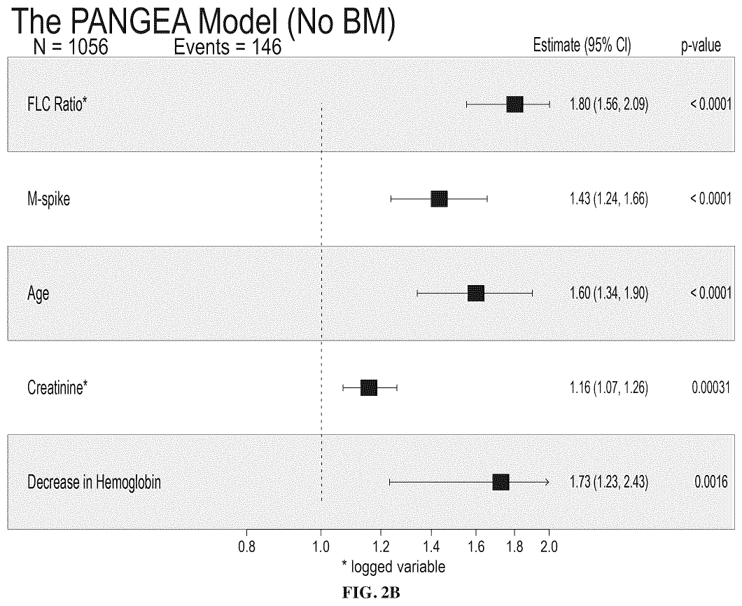

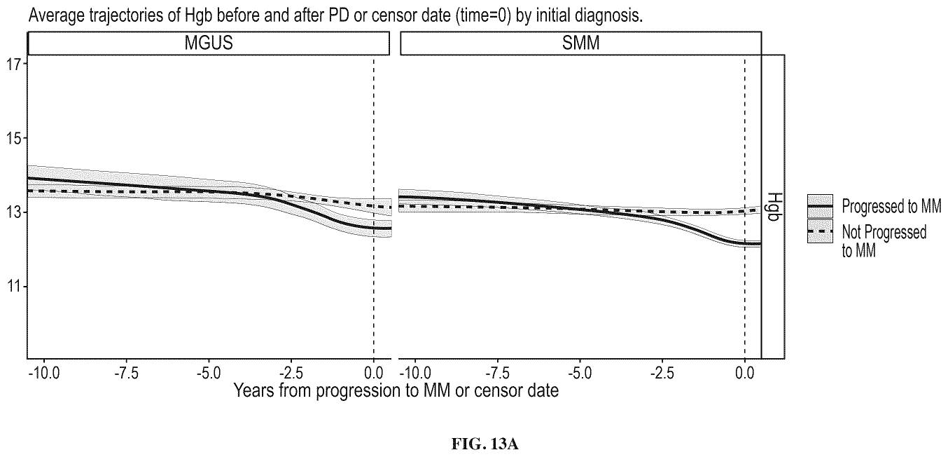

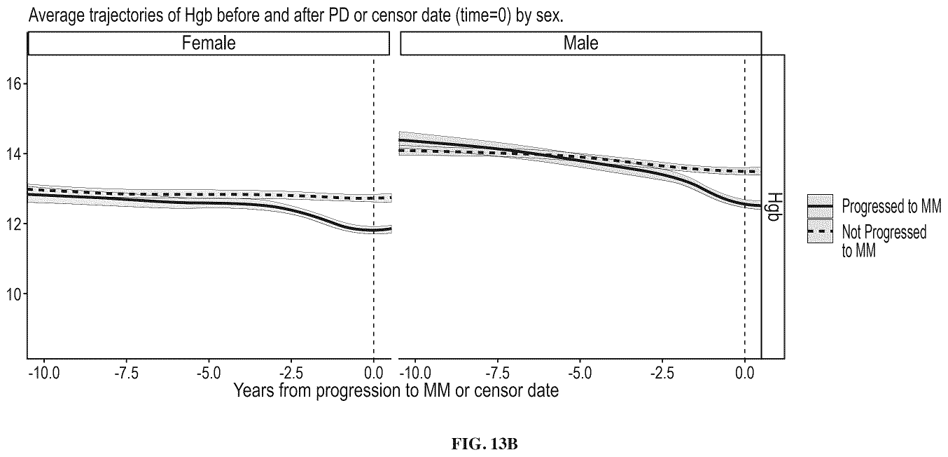

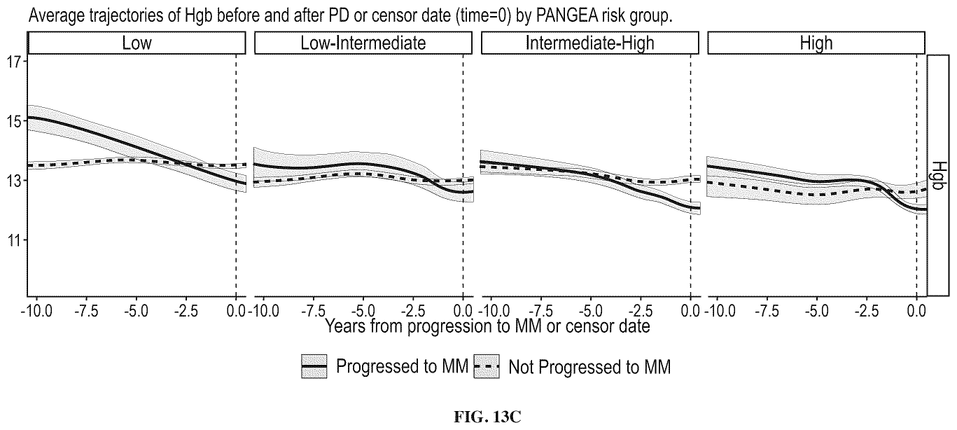

An experiment was undertaken to determine whether age, creatinine, hemoglobin, FLC ratio, M-spike, and BMPC % as continuous variables predicted precursor disease progression to MM (FIGS. 6A-6D). Free light chain (FLC) ratio, M-spike, age, creatinine, and BMPC % and a binary trajectory variable for hemoglobin were used in the PANGEA Model (BM) (FIG. 2A). Specifically, decreases in hemoglobin were significantly associated with decreased risk in the model, and each of the continuous values for FLC ratio, M-spike, age, creatinine, and BMPC % were significant predictors of disease progression. The PANGEA Model (No BM) included the binary trajectory of hemoglobin with FLC ratio, M-spike, age, and creatinine as significant predictors of progression (FIG. 2B). While there was an expected average difference in baseline hemoglobin between males and females (p<0.0001 for patients who did not progress), there was no difference in the rate of change in hemoglobin between male and female patients who did not progress (p=0.830 for the Training Cohort, p=0.220 for the Validation Cohorts) (FIGS. 13A-13C).

The PANGEA Model (BM) and PANGEA Model (No BM) were built directly into an online risk calculator (www.pangeamodels.org) that outputs probabilities of disease progression at years 1, 2, 5, 10, and 25 post-diagnosis.

### Example 3: The PANGEA Model Improved Precursor Progression Risk Analysis Over Standard Models, Regardless of the Incorporation of Bone Marrow (BM) Variables

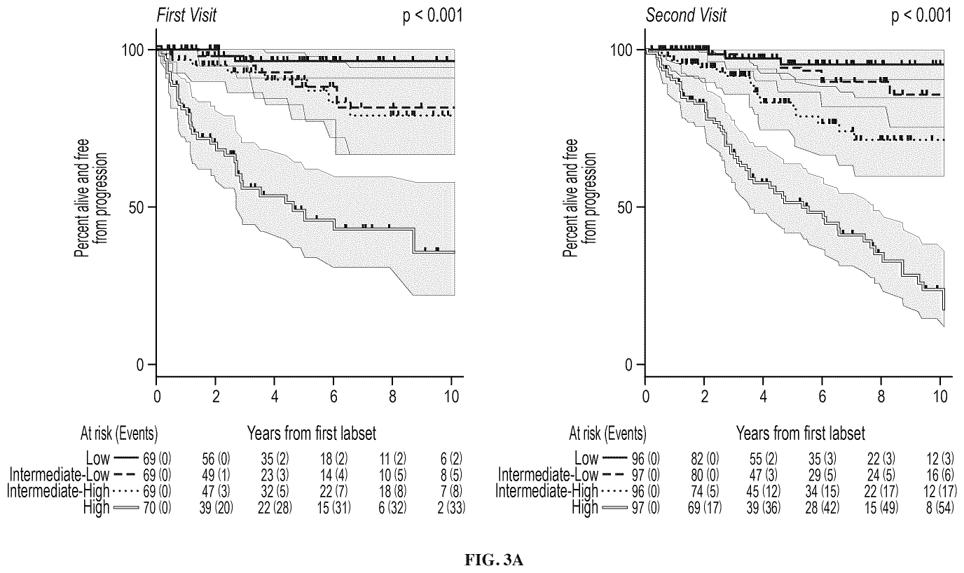

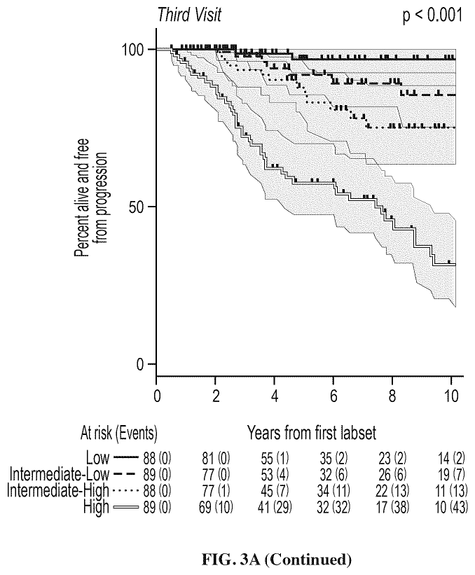

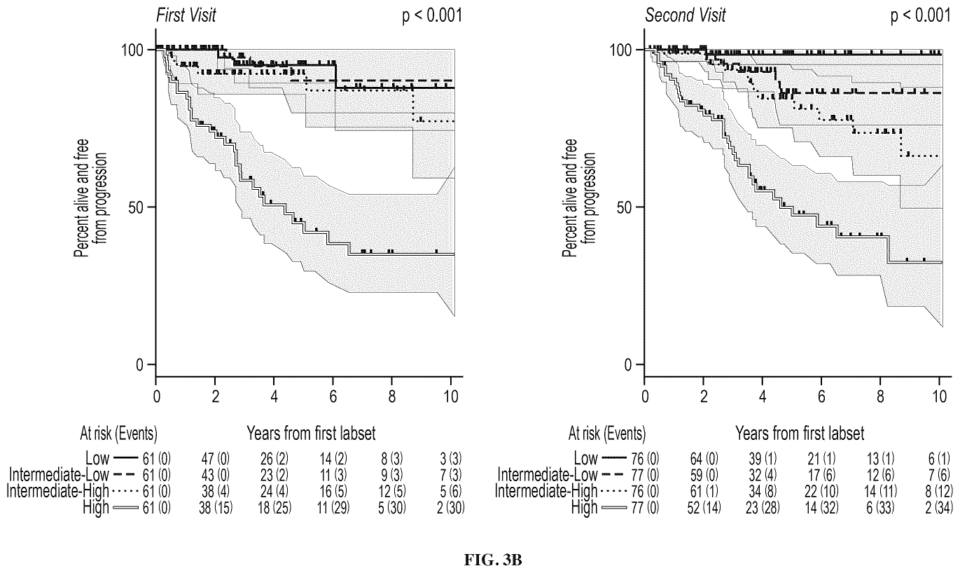

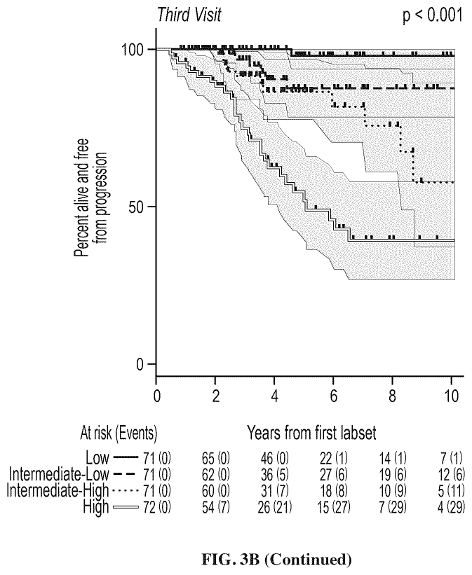

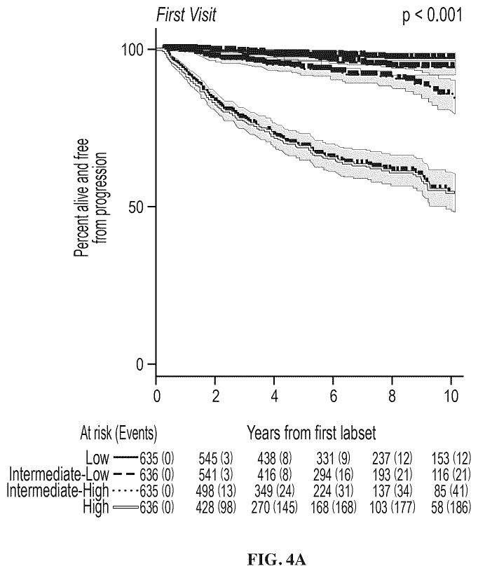

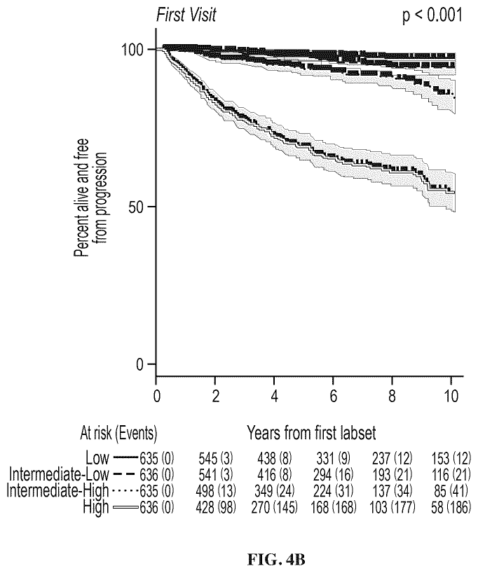

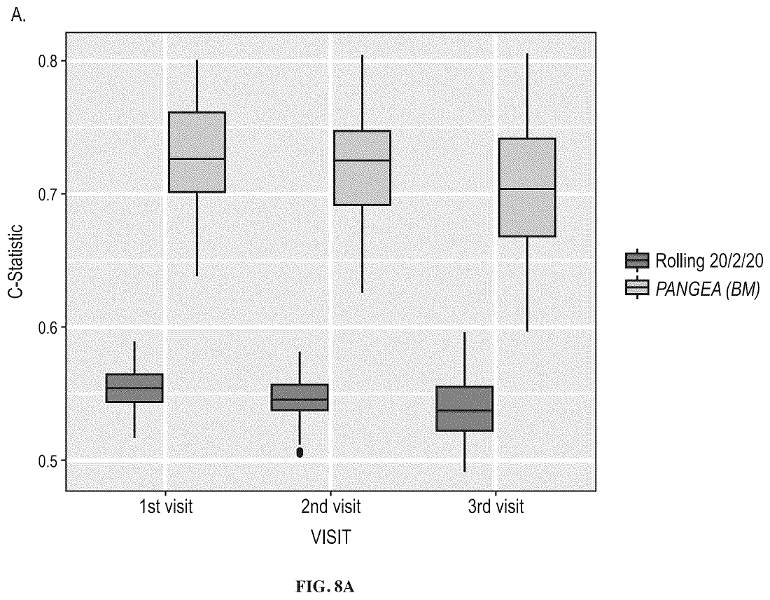

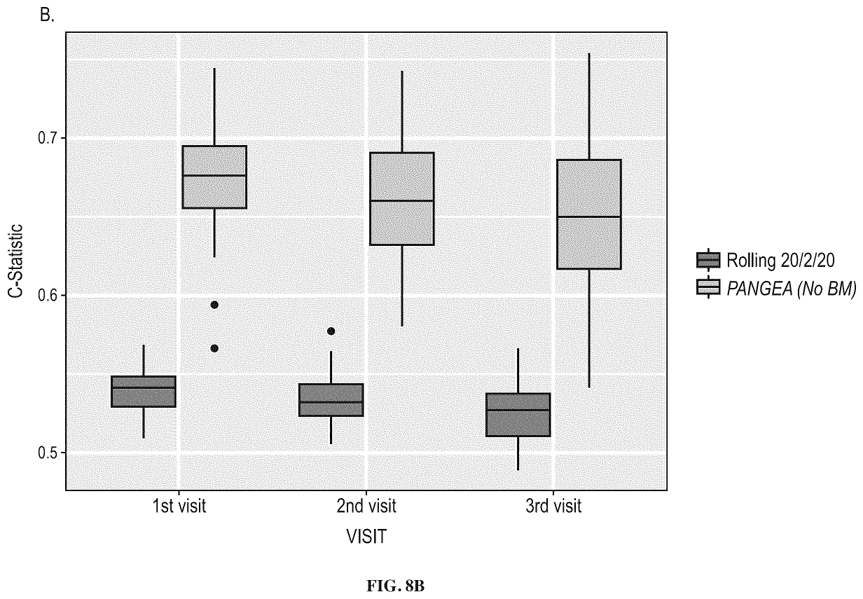

An experiment was undertaken establishing that the PANGEA Model improved prediction of precursor progression compared to both 20/2/20 models (Baseline and Rolling) in Validation Cohort 1 (FIGS. 3A and 3B) and Validation Cohort 2 (FIGS. 4A and 4B), as indicated by a C-statistic increase of more than 10% and minimal change in the corresponding confidence intervals (FIGS. 8A and 8B). The PANGEA (BM) Model showed an increase in C-statistic compared to the Baseline Model of 42% (0.533 (0.480-0.709) to 0.756 (0.629-0.785)) and compared to the Rolling 20/2/20 Model an average increase of 18% (0.613 (0.504-0.704) to 0.720 (0.592-0.775) at Visit 2) and 0.637 (0.386-0.841) to 0.756 (0.547-0.830) at Visit 3)) in Validation Cohort 1 (Table 2). Similarly, the PANGEA (No BM) Model showed a 30% (0.534 (0.501-0.672) to 0.692 (0.614-0.736)) increase in C-statistic compared to the Baseline Model and an average increase of 22% (0.573 (0.518-0.647) to 0.693 (0.605-0.734) at Visit 2 and 0.560 (0.497-0.645) to 0.692 (0.570-0.708) at Visit 3)) compared to the Rolling 20/2/20 Model in Validation Cohort 1 (Table 2). For Validation Cohort 2, predictions were only validated using data at the time of diagnosis, as few patients had longitudinal data. In this cohort, there was a 22% (0.502 (0.472-0.568) to 0.610 (0.525-0.931)) increase (PANGEA Model (BM)) and 45% (0.492 (0.472-0.536) to 0.714 (0.589-0.933)) increase (PANGEA Model (No BM)) in C-statistic compared to both the Baseline Model and Rolling 20/2/20 Model (Table 2).

The PANGEA Models similarly outperformed the Rolling IMWG Model for MGUS patients with improvements of 24% (0.640 (0.518-0.718) to 0.729 (0.643-0.941))) C-statistics by the PANGEA Model (BM) and 31% (0.670 (0.523-0.729) to 0.879 (0.586-0.938))) by the PANGEA Model (No BM) in Validation Cohort 2 (Table 3).

TABLE 2
Performance of the PANGEA Models (BM and No BM) compared to
the Baseline and Rolling Models, measured by C-statistic (95% confidence
interval) as tested in SMM patients of Validation Cohort 1 and Validation
Cohort 2. Confidence intervals demonstrated in FIGS. 8A and 8B.

| | | Baseline Model | Rolling Model | PANGEA |
|---|---|---|---|---|
| | | (20/2/20) | (20/2/20 over time) | Models |
| Validation | BM | 0.533 (0.480- | 0.669 (0.537- | 0.756 (0.629-0.785) |
| Cohort 1: | | 0.709) | 0.696) | |
| Visit 1 | No BM | 0.534 (0.501- | 0.625 (0.526- | 0.692 (0.614-0.736) |
| | | 0.672) | 0.649) | |
| Validation | BM | | 0.613 (0.504- | 0.720 (0.592-0.775) |
| Cohort 1: | | | 0.704) | |
| Visit 2 | No BM | | 0.573 (0.518- | 0.693 (0.605-0.734) |
| | | | 0.647) | |
| Validation | BM | | 0.637 (0.386- | 0.756 (0.547-0.830) |
| Cohort 1: | | | 0.841) | |
| Visit 3 | No BM | | 0.560 (0.497- | 0.692 (0.570-0.708) |
| | | | 0.645) | |
| Validation | BM | 0.502 (0.482- | 0.502 (0.472- | 0.610 (0.525-0.931) |
| Cohort 2: | | 0.604) | 0.568) | |
| Visit 1 | No BM | 0.492 (0.460- | 0.492 (0.472- | 0.714 (0.589-0.933) |
| | | 0.561) | 0.536) | |

TABLE 3
Performance of the PANGEA Models (BM and No BM) compared
to the Rolling IMWG Models, as measured by C-statistic (95%
confidence interval) using MGUS patients of Validation Cohort 2.

| | | BASELINE | ROLLING | PANGEA |
|---|---|---|---|---|
| | | IMWG | IMWG | MODELS |
| VALIDATION | BM | 0.640 (0.500- | 0.640 (0.518- | 0.729 (0.643- |
| COHORT | | 0.807) | 0.718) | 0.941) |
| 2: VISIT 1 | No BM | 0.667 (0.512- | 0.670 (0.523- | 0.879 (0.586- |
| | | 0.836) | 0.729) | 0.938) |

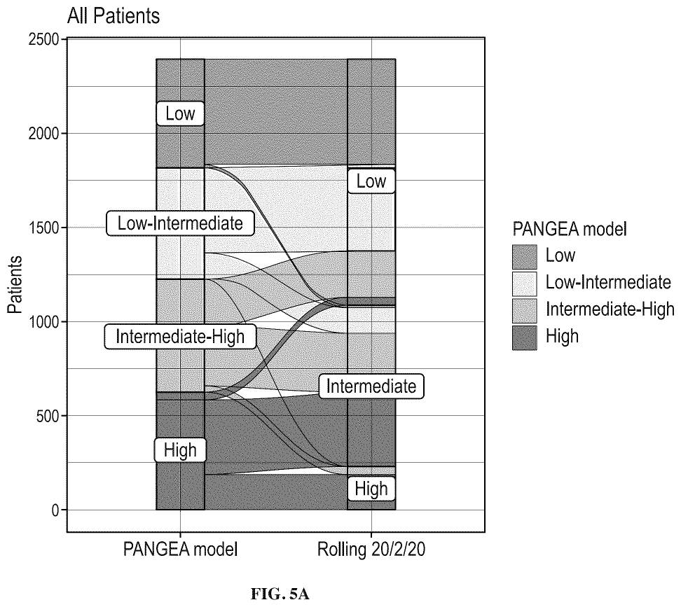

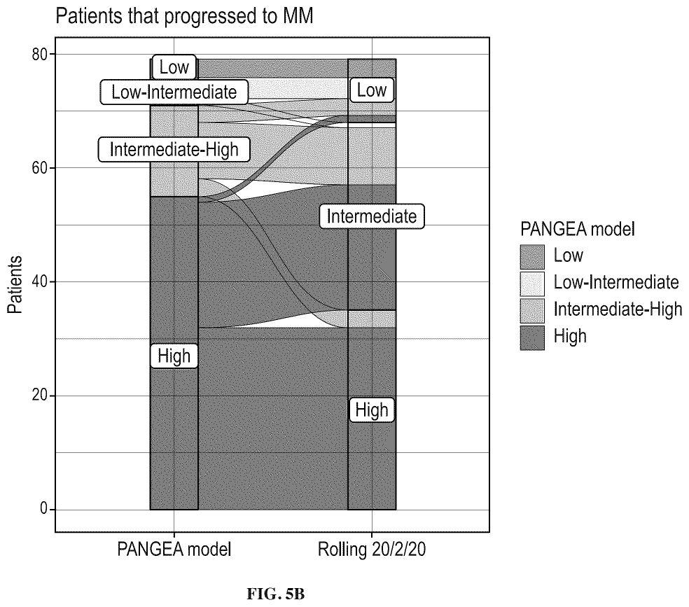

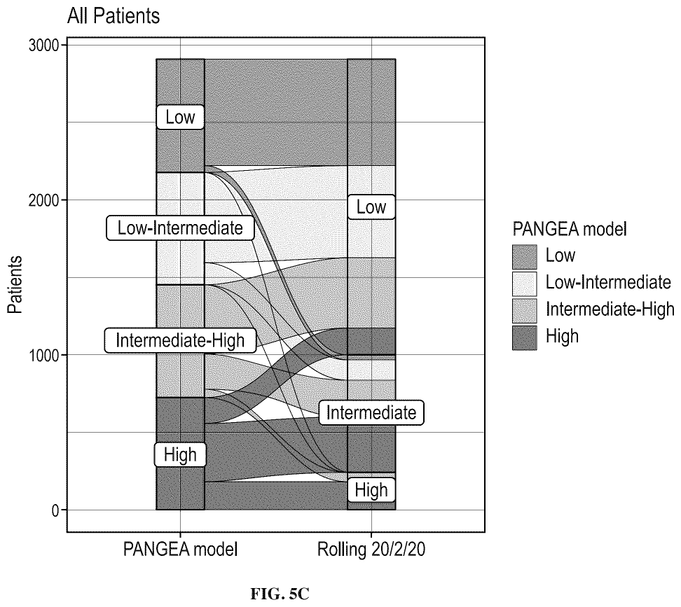

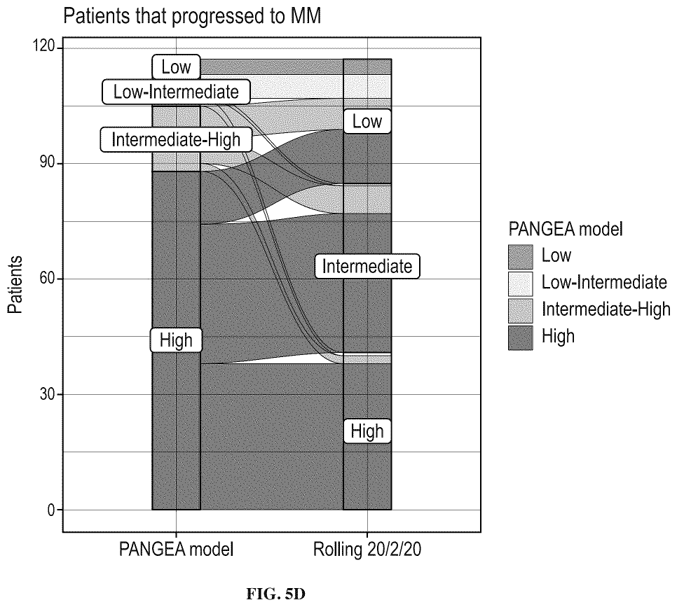

The PANGEA Model outputs probabilities of progression for individual patients. Hence, it was difficult to contrast risk estimates to those of the Baseline and Rolling Models, which stratify patients into risk groups. To allow for direct comparison of risk groups, Validation Cohort 1 (FIGS. 3A and 3B) and Validation Cohort 2 (FIGS. 4A and 4B) were artificially stratified into High, High-Intermediate, Low-Intermediate, and Low progression risk groups using quartiles (0.25, 0.50 and 0.75) of predicted risk. The predicted risk groups for patients were compared in this cohort, and 58% of smoldering multiple myeloma (SMM) patients who eventually progressed to MM were reclassified from a Rolling Model 20/2/20 intermediate-or low-risk category into a PANGEA (BM) high-risk category (FIGS. 5A-5D).

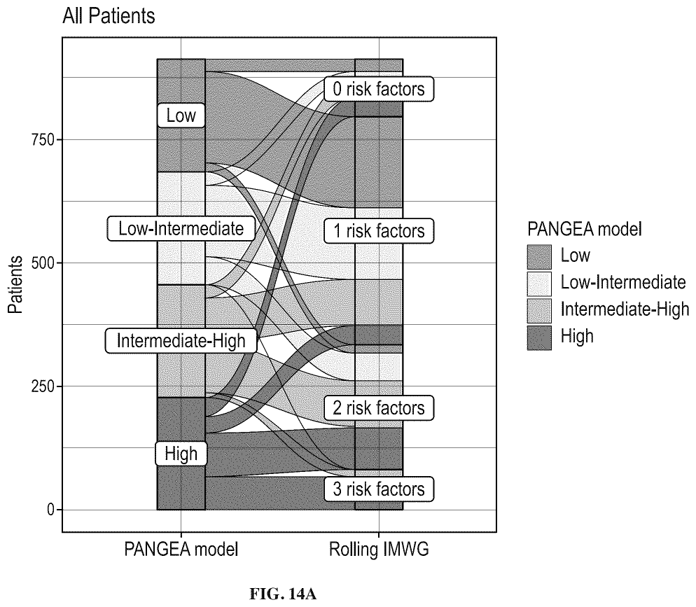

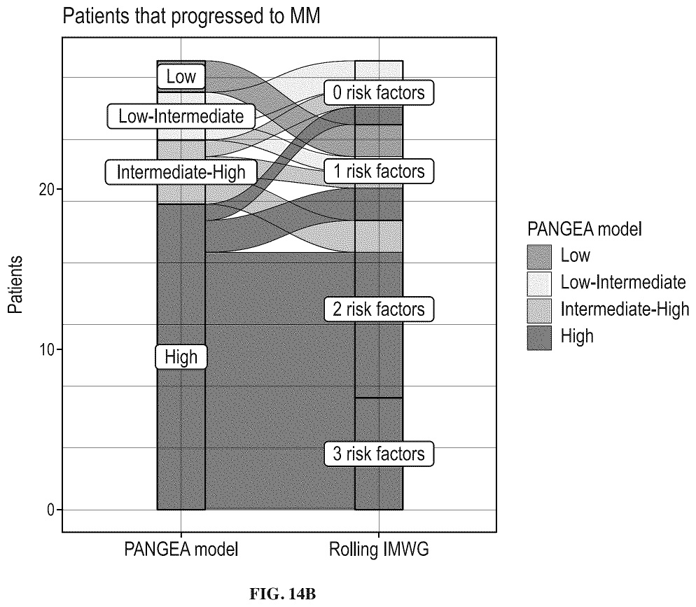

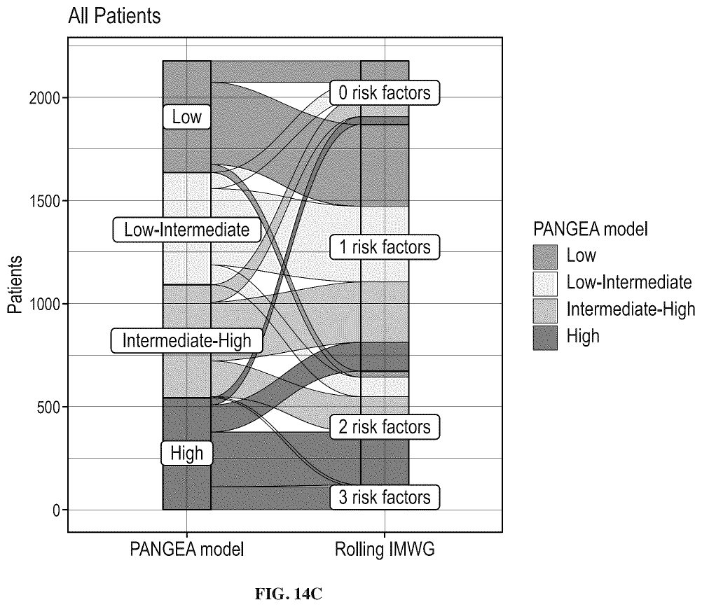

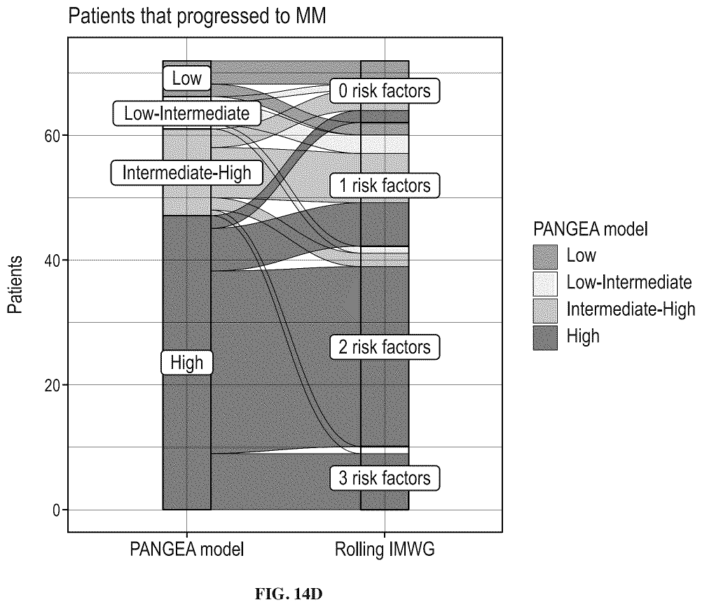

Similarly, 43% of MGUS patients who eventually progressed to MM were reclassified from a Rolling IMWG lower risk category into a PANGEA Model (BM) high-risk category (FIGS. 14A-14D).

### Example 4: High-Risk Cytogenetics Increase Risk of Precursor Disease Progression

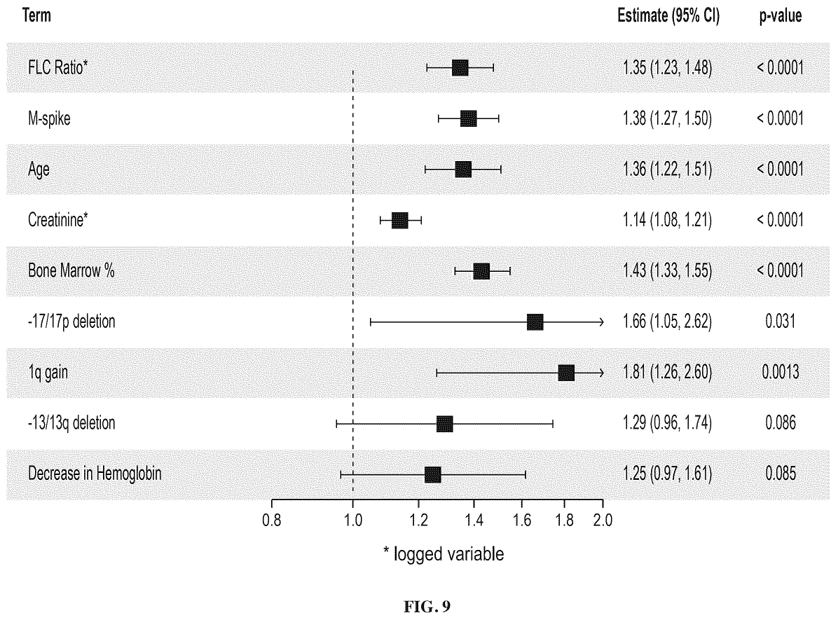

Because enomic aberrations play a critical role in precursor progression, the PANGEA Model (BM) was expanded to include fluorescence in-situ hybridization (FISH) covariates. The resulting PANGEA Model (FISH) used the significant predictors of free light chain (FLC) ratio, M-spike, age, creatinine, and the presence of a chromosome 17 or 17p deletion, 13 or 13q deletion, or 1q gain with binary trajectory of hemoglobin to predict progression risk (FIG. 9). MYC rearrangement (8q24) was also identified as a significant covariate in a subcohort of 957 PANGEA patients from the Training Cohort and Validation Cohort 1 who were tested for this translocation (Table 4). FISH alterations that were not statistically significant covariates included t(11;14), t(4;14), t(6;14), t(14;16), t(14;20) and hyperdiploidy as well as the combined variables of t(4;14), t(14;16), t(6;14), t(14;20) together and t(4;14), t(14;16), 1q gain, −13/13q together.

TABLE 4
Patient Demographics of a PANGEA Subcohort with FISH Analysis.

| | Total | | DFCI | | Greece | | UK | | Czech | | |
|---|---|---|---|---|---|---|---|---|---|---|---|
| | n = 6,445 (%) | | n = 1,219 (19) | | n = 533 (8) | | n = 109 (2) | | n = 4,584 (71) | | p-value |
| t(4; 14) | | | | | | | | | | | |
| No | 899 | (14) | 874 | (72) | — | | 25 | (23) | — | | <0.001\hat{ } |
| Yes | 117 | (2) | 62 | (5) | 9 | (2) | 3 | (3) | 43 | (1) | |
| Missing | 5,429 | (84) | 283 | (23) | 524 | (98) | 81 | (74) | 4,541 | (99) | |
| t(6; 14) | | | | | | | | | | | |
| No | 946 | (15) | 921 | (76) | — | | 25 | (23) | — | | <0.001\hat{ } |
| Yes | 14 | (0) | 11 | (1) | — | | — | | 3 | (0) | |
| Missing | 5,485 | (85) | 287 | (24) | 533 | (100) | 84 | (77) | 4,581 | (100) | |
| t(11; 14) | | | | | | | | | | | |
| No | 723 | (11) | 701 | (58) | — | | 22 | (20) | — | | <0.001\hat{ } |
| Yes | 338 | (5) | 245 | (20) | 4 | (1) | 12 | (11) | 77 | (2) | |
| Missing | 5,384 | (84) | 273 | (22) | 529 | (99) | 75 | (69) | 4,507 | (98) | |
| t(14; 16) | | | | | | | | | | | |
| No | 913 | (14) | 890 | (73) | — | | 23 | (21) | — | | <0.001\hat{ } |
| Yes | 62 | (1) | 45 | (4) | 3 | (1) | 4 | (4) | 10 | (0) | |
| Missing | 5,470 | (85) | 284 | (23) | 530 | (99) | 82 | (75) | 4,574 | (100) | |
| t(14; 20) | | | | | | | | | | | |
| No | 935 | (15) | 910 | (75) | — | | 25 | (23) | — | | 0.47\hat{ } |
| Yes | 19 | (0) | 19 | (2) | — | | — | | — | | |
| Missing | 5,491 | (85) | 290 | (24) | 533 | (100) | 84 | (77) | 4,584 | (100) | |
| t(14; 18) | | | | | | | | | | | |
| No | 950 | (15) | 925 | (76) | — | | 25 | (23) | — | | 0.74\hat{ } |
| Yes | 4 | (0) | 4 | (0) | — | | — | | — | | |
| Missing | 5,491 | (85) | 290 | (24) | 533 | (100) | 84 | (77) | 4,584 | (100) | |
| −17/17p deletion | | | | | | | | | | | |
| No | 885 | (14) | 860 | (71) | — | | 25 | (23) | — | | <0.001\hat{ } |
| Yes | 132 | (2) | 82 | (7) | 2 | (0) | — | | 48 | (1) | |
| Missing | 5,428 | (84) | 277 | (23) | 531 | (100) | 84 | (77) | 4,536 | (99) | |
| 6q deletion | | | | | | | | | | | |
| No | 949 | (15) | 924 | (76) | — | | 25 | (23) | — | | 0.69\hat{ } |
| Yes | 6 | (0) | 6 | (0) | — | | — | | — | | |
| Missing | 5,490 | (85) | 289 | (24) | 533 | (100) | 84 | (77) | 4,584 | (100) | |
| deletion 11q22 | | | | | | | | | | | |
| No | 952 | (15) | 927 | (76) | — | | 25 | (23) | — | | 0.006\hat{ } |
| Yes | 4 | (0) | 3 | (0) | — | | 1 | (1) | — | | |
| Missing | 5,489 | (85) | 289 | (24) | 533 | (100) | 83 | (76) | 4,584 | (100) | |
| 1q gain | | | | | | | | | | | |
| No | 843 | (13) | 823 | (68) | — | | 20 | (18) | — | | <0.001\hat{ } |
| Yes | 296 | (5) | 113 | (9) | 14 | (3) | 16 | (15) | 153 | (3) | |
| Missing | 5,306 | (82) | 283 | (23) | 519 | (97) | 73 | (67) | 4,431 | (97) | |
| 8q24/MYC rearrangement | | | | | | | | | | | |
| No | 937 | (15) | 912 | (75) | — | | 25 | (23) | — | | 0.53\hat{ } |
| Yes | 20 | (0) | 19 | (2) | — | | 1 | (1) | — | | |
| Missing | 5,488 | (85) | 288 | (24) | 533 | (100) | 83 | (76) | 4,584 | (100) | |
| −13/13q deletion | | | | | | | | | | | |
| No | 774 | (12) | 751 | (62) | — | | 23 | (21) | — | | <0.001\hat{ } |
| Yes | 423 | (7) | 188 | (15) | 27 | (5) | 6 | (6) | 202 | (4) | |
| Missing | 5,248 | (81) | 280 | (23) | 506 | (95) | 80 | (73) | 4,382 | (96) | |
| +3/+7 hyperdiploid | | | | | | | | | | | |
| No | 866 | (13) | 841 | (69) | — | | 25 | (23) | — | | 0.30\hat{ } |
| Yes | 94 | (1) | 93 | (8) | — | | 1 | (1) | — | | |
| Missing | 5,485 | (85) | 285 | (23) | 533 | (100) | 83 | (76) | 4,584 | (100) | |
| +9/+15 hyperdiploid | | | | | | | | | | | |
| No | 805 | (12) | 780 | (64) | — | | 25 | (23) | — | | 0.08\hat{ } |
| Yes | 158 | (2) | 157 | (13) | — | | 1 | (1) | — | | |
| Missing | 5,482 | (85) | 282 | (23) | 533 | (100) | 83 | (76) | 4,584 | (100) | |
| trisomy 4 | | | | | | | | | | | |
| No | 949 | (15) | 924 | (76) | — | | 25 | (23) | — | | 0.69\hat{ } |
| Yes | 6 | (0) | 6 | (0) | — | | — | | — | | |
| Missing | 5,490 | (85) | 289 | (24) | 533 | (100) | 84 | (77) | 4,584 | (100) | |
| trisomy 12 | | | | | | | | | | | |
| No | 951 | (15) | 926 | (76) | — | | 25 | (23) | — | | 0.74\hat{ } |
| Yes | 4 | (0) | 4 | (0) | — | | — | | — | | |
| Missing | 5,490 | (85) | 289 | (24) | 533 | (100) | 84 | (77) | 4,584 | (100) | |
| trisomy 18 | | | | | | | | | | | |
| No | 949 | (15) | 925 | (76) | — | | 24 | (22) | — | | 0.03\hat{ } |
| Yes | 6 | (0) | 5 | (0) | — | | 1 | (1) | — | | |
| Missing | 5,490 | (85) | 289 | (24) | 533 | (100) | 84 | (77) | 4,584 | (100) | |

\hat{ }Cochran-Armitage test

The importance of precursor disease in the development of multiple myeloma (MM) has led to the creation of stratification systems which identify patients at the highest risk of progressing to more advanced disease. However, current monoclonal gammopathy of undetermined significance (MGUS)/smoldering multiple myeloma (SMM) progression prediction algorithms stratify patients into risk groups using baseline measurements rather than longitudinal, time-varying markers. Additionally, leading models such as Mayo's 20/2/20 model (Lakshman A, et al., “Risk stratification of smoldering multiple myeloma incorporating revised IMWG diagnostic criteria,” Blood Cancer J 8:59, (2018)) and the Spanish PETHEMA criteria (Pérez-Persona E, et al., “New criteria to identify risk of progression in monoclonal gammopathy of uncertain significance and smoldering multiple myeloma based on multiparameter flow cytometry analysis of bone marrow plasma cells,” Blood 110:2586-2592 (2007)) failed to agree on which precursor patients classified as high-risk (Joseph NS, et al. “The Role of Early Intervention in High-Risk Smoldering Myeloma,” Am Soc Clin Oncol Educ Book 40:1-9 (2020)). Discordant definitions of disease risk and an inability to update this risk over time have led to major differences in the inclusion criteria of clinical trials and treatment strategies for precursor patients. The advent of personalized medicine offers opportunities to evaluate progression risk with statistical models and translate time-varying markers into predictions that support clinical decisions.

A cohort of precursor patients with extensive longitudinal data was assembled to develop the PANGEA Model, a multivariate Cox regression that uses widely-available, time-varying markers with and without bone marrow (BM) data to improve predictions of precursor progression risk at the individual level. The PANGEA Model incorporated clinical variables beyond typical measures of tumor burden, including creatinine, age, and hemoglobin in addition to those in the 20/2/20 criteria (M-spike, serum kappa free light chain (FLC) ratio, and bone marrow plasma cell percent (BMPC %)). The PANGEA models (with bone marrow [BM] data and without bone marrow [no BM] data) were compared to current criteria (International Myeloma Working Group [IMWG] monoclonal gammopathy of undetermined significance and 20/2/20 smoldering multiple myeloma risk criteria. A difference between the PANGEA Model and the 20/2/20 risk criteria was the ability of the PANGEA Model to provide patient-specific probabilities of progression starting from disease development. This means that the PANGEA Model was less influenced by inconsequential changes in markers, which can occur within limited periods and can confound criteria that define risk at a single time point. The PANGEA Model allowed for personalized prognostication and demonstrated a significant precision improvement over current risk criteria as well.

When different models were applied to the same cohort, C-statistics allowed for direct comparison of predictive accuracy. Analysis of the PANGEA Model compared to the Baseline and Rolling 20/2/20 Models demonstrated changes in C-statistic >10%. This dramatic increase in C-statistic was clinically validated by the improved accuracy in early identification of patients who later progressed to overt MM with 58% of progressors identified as high-risk by the PANGEA Model and not the Rolling 20/2/20 Model (FIGS. 5A-5D). Combined, these findings highlighted that the PANGEA Model was both appropriate and necessary when describing changes in disease risk after diagnosis.

A goal of the experiments presented herein was to determine the role of bone marrow biopsy (BMbx) in risk prediction. Despite the reliance of current stratification models on bone marrow plasma cell percent (BMPC %) as a progression predictor, many precursor patients do not regularly undergo bone marrow biopsies (BMbxs) or forgo them altogether. These patients thus cannot be adequately assessed by risk criteria that rely on BMPC %. The PANGEA Model (No BM) demonstrated that progression risk could be accurately estimated using trends in serum markers. Specifically, both PANGEA Models (BM and No BM) outperformed the Baseline and Dynamic Models (change in C-statistic >10%) with the PANGEA Model (BM) only slightly outperforming the PANGEA Model (No-BM) (change in C-statistic <10%) in Validation Cohort 1, and the PANGEA Model (No BM) outperforming the PANGEA Model (No-BM) (change in C-statistic <10%) in Validation Cohort 2 (Table 2). This suggested that variables derived from BMbxs were not required to accurately determine progression risk when other factors were considered. When BMbx data is no longer required to determine progression risk, the distinction between MGUS and SMM is blurred. It is therefore advisable to transition from the division of patients with precursor conditions into a few risk groups (MGUS vs. SMM and SMM risk groups) to a granular segmentation of the precursor population at the individual level for improved precision medicine. Thus, regardless of a patient's BM status, the PANGEA Model could be used via an online PANGEA App to easily calculate patient risk in the clinic as both models (i.e., with and without bone marrow biopsy) improved upon current stratification algorithms (www.pangeamodels.org).

Genomic and epigenetic factors that lead to progression to multiple myeloma (MM) are also a critical part of a patient's progression risk profile. Therefore, the PANGEA Model (FISH) was developed, which allows for the potential incorporation of sequential cytogenetic data in personalized risk prediction. The PANGEA Model (FISH) was innovative in that it examined changes in cytogenetic alterations when providing probabilities of disease progression. Patients used to build the PANGEA Model (FISH) included those with singular and multiple cytogenetic abnormalities throughout disease evolution such that as high-risk (−13/13q, −17/17p, +1q) abnormalities accumulated in patients' bone marrow, their risk of progression worsened. This demonstrated the value of modeling dynamic FISH variables and suggested that previously imperceptible clonal tumor evolution may be approximated by clinical cytogenetic results.

The PANGEA Model can dramatically improve how clinicians inform patients of risk of developing myeloma and aid in the decision-making process for early therapeutic interception, particularly when recommending follow-up testing to allow for monitoring of time-varying markers. The PANGEA Model is accessible, using variables available in all clinical settings, enabling it to be used at the individual patient level as well as in clinical trials to allow harmonization across studies and for the rapid development of therapeutic interventions.

The following methods were employed in the above examples.

### The PANGEA Project: One Training Cohort and Two Independent Validation Cohorts

PANGEA was an international cohort of precursor patients with serial clinical and biological variables (Tables 5 and 6). Patients ≥18 years of age were identified retrospectively from Nov. 17, 2019 to Apr. 13, 2022 at oncology centers (Dana-Farber Cancer Institute (DFCI, Boston, MA, USA), National and Kapodistrian University of Athens (Athens, Greece), University College London (UCL; London, UK)) and the cancer group Registry of Monoclonal Gammopathies (RMG) (Czech Republic).

The PANGEA Project included the Training Cohort containing 1217 (715 MGUS and 502 SMM)

DFCI patients; Validation Cohort 1 contained 642 (143 MGUS and 390 SMM) University of Athens patients and 109 SMM UCL patients, and Validation Cohort 2 contained 4582 (4073 MGUS and 509 SMM, with 745 progressing to multiple myeloma) RMG patients (FIG. 1, Table 1). The median number of timepoints (clinic visits) was seven (range one to 40) for the training cohort, six (range one to 40) for the validation cohort 1, and one (range one to one) for validation cohort 2. The median follow-up time was 4·2 (IQR 0·0-30·5) years for the training cohort, 2·9 (IQR 0·0-21·4) years for the validation cohort 1, and 3·6 (IQR 0·0-73·9) years for validation cohort 2. Validation cohort 1 had a similar progression proportion (2·23% [95% CI 1·19-3·79]), defined as the proportion of patients who progressed to multiple myeloma within three clinical visits, to the training cohort (2-18% [1·35-3·10]), whereas validation cohort 2 had a lower proportion of those who had disease progression (0-11% [0·03-0·28]). Patient information was collected for total protein, IgA via nephelometry, IgM, IgG, kappa free light chain (FLC) and lambda FLC via Optilite® (Binding Site), FLC ratio involved/uninvolved, calcium, creatinine, albumin, hemoglobin, LDH, beta-2 microglobulin, M-spike, and weight. Serial values were annotated on average at 5-month (IQR 3-8 months) time intervals from the date of MGUS or SMM diagnosis, censoring at the date of progression to active MM, last follow-up, initiation of precursor treatment, or death. Gender, race, ethnicity, age at diagnosis, height, progression, survival status, immunofixation isotype, and bisphosphonate use were also collected. For all BMbx, plasma cell percentages were collected from core biopsies and FISH results from bone marrow aspirates. Patients diagnosed with overt multiple myeloma at diagnosis were excluded from analysis, and patients treated with therapy during their precursor disease course were censored at treatment start dates. Patients were included in analysis until the date of progression per SLIM-CRAB criteria, death, or initiation of treatment. In all three cohorts, patients were selected for analysis from tissue-banking and retrospective monitoring trials for precursor disease states.

TABLE 5
Patient Demographics of Training and Validation Cohorts of the
PANGEA Project.

| | | Validation | |
|---|---|---|---|
| | Training | Cohort 1 | Validation |
| | Cohort | (Greece, | Cohort 2 |
| Total | (DFCI) | UK) | (Czech) |
| n = 6,441 | n = 1,217 | n = 642 | n = 4,582 |
| (%) | (19) | (10) | (71) |

Age at initial diagnosis (years)

| | | | | |
|---|---|---|---|---|
| Median (range) | 64.22 (19.52- | 62.00 (22.00- | 64.00 (28.50- | 65.21 (19.52- |
| | 94.00) | 94.00) | 89.09) | 93.77) |
| Missing | 42 (1) | — | — | 42 (1) |

Number of patient labsets

| | | | | |
|---|---|---|---|---|
| Median (range) | 7 (1-40) | 7 (1-40) | 6 (1-40) | 1 (1-1) |

Interval between visits (months)

| | | | | |
|---|---|---|---|---|
| Median (range) | 5 (0-140) | 6 (0-140) | 5 (0-103) | 5 (0-112) |

Sex

| | | | | |
|---|---|---|---|---|
| Female | 3,430 (53) | 642 (53) | 374 (58) | 2,414 (53) |
| Male | 3,009 (47) | 575 (47) | 266 (41) | 2,168 (47) |
| Missing | 2 (0) | — | 2 (0) | — |

Race

| | | | | |
|---|---|---|---|---|
| White | 1,575 (24) | 992 (82) | 583 (91) | — |
| Black or African | 156 (2) | 137 (11) | 19 (3) | — |
| American | | | | |
| Asian | 45 (1) | 28 (2) | 17 (3) | — |
| Multiple | 7 (0) | 6 (0) | 1 (0) | — |
| Declined | 9 (0) | 8 (1) | 1 (0) | — |
| Other | 37 (1) | 26 (2) | 11 (2) | — |
| Missing | 4,612 (72) | 20 (2) | 10 (2) | 4,582 (100) |

Ethnicity

| | | | | |
|---|---|---|---|---|
| Declined | 8 (0) | 8 (1) | — | — |
| Not Hispanic or | 1,053 (16) | 1,052 (86) | 1 (0) | — |
| Latino | | | | |
| Hispanic or | 54 (1) | 54 (4) | — | — |
| Latino | | | | |
| Missing | 5,326 (83) | 103 (8) | 641 (100) | 4,582 (100) |

Original diagnosis

| | | | | |
|---|---|---|---|---|
| MGUS | 4,931 (77) | 715 (59) | 143 (22) | 4,073 (89) |
| SMM | 1,510 (23) | 502 (41) | 499 (78) | 509 (11) |

Progression to SMM

| | | | | |
|---|---|---|---|---|
| Not progressed | 4,520 (70) | 437 (36) | 138 (21) | 3,945 (86) |
| to SMM | | | | |
| Progressed | 411 (6) | 278 (23) | 5 (1) | 128 (3) |
| to SMM | | | | |
| SMM as original | 1,510 (23) | 502 (41) | 499 (78) | 509 (11) |
| diagnosis | | | | |

Progression to MM

| | | | | |
|---|---|---|---|---|
| Not Progressed | 5,381 (84) | 1,045 (86) | 499 (78) | 3,837 (84) |
| to MM | | | | |
| Progressed | 1,060 (16) | 172 (14) | 143 (22) | 745 (16) |
| to MM | | | | |

Immunofixation

| | | | | |
|---|---|---|---|---|
| IgG | 4,908 (76) | 882 (72) | 462 (72) | 3,564 (78) |
| IgA | 1,127 (17) | 232 (19) | 149 (23) | 746 (16) |
| Light Chain | 179 (3) | 75 (6) | 14 (2) | 90 (2) |
| Only | | | | |
| Biclonal | 34 (1) | 21 (2) | 9 (1) | 4 (0) |
| Missing | 193 (3) | 7 (1) | 8 (1) | 178 (4) |

Died

| | | | | |
|---|---|---|---|---|
| No | 5,080 (79) | 1,133544 (93) | 569 (89) | 3,405 (74) |
| Yes | 1,334 (21) | 84 (7) | 73 (11) | 1,177 (26) |

Censored for Treatment

| | | | | |
|---|---|---|---|---|
| Yes | 229 (4) | 222 (18) | 6 (1) | 1 (0) |
| No | 1,370 (21) | 995 (82) | 636 (99) | — |

TABLE 6
Patient Demographics of Training and Validation Cohorts of the
PANGEA Project. In the table “MGUS” represents
“monoclonal gammopathy of undetermined significance,” “SMM”
represents “smoldering multiple myeloma,” “MM” represents
“multiple myeloma,” and “IQR” represents “interquartile range.”

| | 20/2/20 RISK GROUPS | | |
|---|---|---|---|
| Total | LOW | INTERMEDIATE | HIGH |
| n = 1,218 (%) | RISK SMM | RISK SMM | RISK SMM |

Visit 1 Diagnosis

| | | | | |
|---|---|---|---|---|
| MGUS | 373 (31) | — | — | — |
| SMM | 845 (69) | 635 (75) | 128 (15) | 82 (10) |
| MM | 0 (0) | — | — | — |

Visit 2 Diagnosis

| | | | | |
|---|---|---|---|---|
| MGUS | 373 (31) | — | — | — |
| SMM | 837 (68) | 630 (75) | 128 (15) | 79 (10) |
| MM | 8 (1) | — | — | — |

Visit 3 Diagnosis

| | | | | |
|---|---|---|---|---|
| MGUS | 370 (30) | — | — | — |
| SMM | 823 (68) | 620 (75) | 126 (15) | 78 (10) |
| MM | 25 (2) | — | — | — |

Time Between Visit 1 & Visit 2 (months)

| | | | | |
|---|---|---|---|---|
| Median (IQR) | 2.9 (0.9-7.8) | 1.3 (0.4-2.7) | 1.2 (0.5-2.5) | 1.0 (0.4-2.5) |

Time Between Visit 2 & Visit 3 (months)

| | | | | |
|---|---|---|---|---|
| Median (IQR) | 5.9 (2.6-9.2) | 4.1 (1.6-6.4) | 3.7 (0.9-6.1) | 2.2 (0.9-5.6) |

### Clinical Annotation

Patient information was collected by electronic medical record review for myeloma-specific variables, including total protein, IgA, IgM, IgG, kappa free light chain (FLC), lambda FLC, FLC ratio involved/uninvolved, calcium, creatinine, albumin, hemoglobin, LDH, beta-2 microglobulin, M-spike, and weight. The time of diagnosis and the first visit coincided in all cohorts (i.e., the average time between date of original diagnosis and visit 1 was 0 months for training cohort, validation cohort 1, and validation cohort 2). Serial values were annotated on average at 5-month time intervals from the date of MGUS or SMM diagnosis, stopping at the date of progression to active MM or censoring at the date of last follow-up, initiation of precursor treatment, or death. Gender, race, ethnicity, age at diagnosis, height, progression, survival status, immunofixation isotype, and bisphosphonate use were also collected.

For all bone marrow biopsies (BMbxs), plasma cell percentage and FISH findings were abstracted from medical records. Specifically, the following probes were considered for analysis and annotated from the pathology note as either positive, negative, or failed: t(4;14); t(6;14), t(11;14), t(14;16), t(14;20), t(14;18), −17/17p deletion, 6q deletion, 11q22 deletion, 1q gain, 8q24/MYC rearrangement, −13/13q deletion, +3/+7 hyperdiploid, +9/+15 hyperdiploid, trisomy 4, trisomy 12, and trisomy 18. Validation Cohort 2 did not use probes for 8q24/MYC rearrangements on patient biopsies.

### Prediction Modeling

The PANGEA Model, a multivariate Cox regression with time-varying markers, was built by selecting significant progression predictors (age, FLC ratio, M-spike, creatinine, bone marrow plasma cell percent (BMPC %)) and hemoglobin trajectories identified across all MGUS and SMM patients of the Training Cohort. Two variables, FLC ratio and creatinine, were log-transformed to reduce the effect of outliers in estimating the parameters of the model. The model assumed that the hazard (a measure of short-term risk) of progression to MM was a linear function which only depends on a patient's profile.

To incorporate serial measurements and model trajectories of clinical markers, linear regressions were fit to individual lab values at each visit date using data from a given visit and all prior visits. For each regression, the significance of the t-test was evaluated for the slope, and for regression results with p<0.1 and a positive slope, a trajectory indicator variable (trajij for variable i at timepoint j) was set to 1,and 0 otherwise. As this regression required sufficient data to fit a linear regression, the trajectory variables were set to 0 for the first two timepoints for all variables for each patient. The trajectory variables were then computed at each time point for each lab value, and the trajectory variables were added as time-varying predictors to the Cox model. Only the increasing trajectory variable for hemoglobin was significant. Finally, predictors with a Ward test p<0.01 were removed, leaving a continuous time-varying Cox model with age, kappa free light chain (FLC) ratio, M-spike, creatinine, bone marrow plasma cell percent (BMPC %) and the trajectory variable, traj_{hemoglobin}. This was the PANGEA Model (FIG. 2A). Then all markers that required a bone marrow biopsy (BMbx) were removed and the modeling process was repeated to produce the PANGEA Model (No BM) with four continuous predictors (age, FLC ratio, M-spike, creatinine) and the trajectory variable, traj_{hemoglobin} (FIG. 2B). Finally, to visualize the time to progression for patients in the validation cohorts, patients in Validation Cohort 1 and 2 were divided into quartiles (Low, Intermediate-Low, Intermediate-High and High) based on their predicted risk from the PANGEA Model (BM) or the PANGEA Model (No BM). Predicted risk was estimated in the validation cohorts using data inclusive of Visit 1, 2 or 3 for Validation Cohort 1 and data from Visit 1 (i.e., baseline) for Validation Cohort 2. The progression for these groups of patients was visualized using Kaplan-Meier curves for time to progression or death (with patients censored at time of treatment) for patients who qualified for the PANGEA Model by having all necessary marker values available at the visit of interest (FIGS. 3A and 3B and FIGS. 4A and 4B).

### PANGEA Model Validation

Risk scores were used to calculate C-statistics for Validation Cohort 1 and 2 to compare the performance of the PANGEA Model (BM and No BM) to current risk stratification criteria. The C-statistic is a standard metric used to compare prediction models with a C-statistic of 0.5 indicating that a model performs no better than random chance and a C-statistic of 1 indicating correct prediction of every individual. For the PANGEA Model (BM and No BM), hazard ratios were fit using the data from the Training Cohort, and the Cox linear predictor was computed using the data for each of the Validation Cohorts, and C-statistics at Visits 1, 2, and 3 using data was computed from the respective visit and prior visits (Table 2, Table 3).

The average number of timepoints for Validation Cohort 1 was 6 and for Validation Cohort 2 was 1 (Table 5); thus, Validation Cohort 1 was used to validate how the PANGEA Model performed for patients with long follow-up and Validation Cohort 2 to validate how the PANGEA Model performed at diagnosis (Visit 1). When comparing to current risk stratification criteria, application of the 20/2/20 criteria as binary cutoffs at diagnosis was referred to as the Baseline Model and re-stratification by the 20/2/20 criteria as discrete variables over time as the Rolling Model. For the Baseline Model, the Cox model was fit in the Training Cohort to estimate the hazard ratios for risk groups and the Cox linear predictor was computed in the Validation Cohorts, and the Validation Cohort C-statistic was computed. For the Rolling Model, a time-varying Cox model was fit in the Training Cohort to estimate hazard ratios of the model, and the linear predictor and model C-statistic at Visit 1, 2, and 3 in Validation Cohort 1 was computed (Table 2).

### Fluorescence In Situ Hybridization (FISH) Prediction Modeling

Due to the regular absence and/or failure of FISH testing on bone marrow biopsy (BMbx) and rarity of some cytogenetic alterations, the cohort had a limited number of FISH results. To ensure statistical power, predictive modeling with FISH findings was conducted on a combined cohort of patients from the PANGEA Training Cohort, Validation Cohort 1, and Validation Cohort 2. Patients were selected with one or more successful FISH panels and corresponding laboratory datasets, resulting in a subcohort of patients (Table 4). The PANGEA Model (FISH) was then built using clinical markers that were significant progression predictors (age, kappa free light chain (FLC) ratio, M-spike, creatinine, bone marrow plasma cell percent (BMPC %), −17/17p deletion, 1q gain, −13/13q deletion) together with the significant binary trajectory variable of increasing hemoglobin, which was protective.

### Clinical Calculator

A web application was developed that allowed input of patient values for variables (age, FLC ratio, M-spike, creatinine, hemoglobin, and, optionally, BMPC %) of the PANGEA Model (BM and No BM) and produced progression risk predictions (www.pangeamodels.org). The resulting PANGEA App output a patient's risk of progression using these markers.

### PANGEA Training Cohort

To develop a model that could predict progression to MM, Cox regressions were first trained on the PANGEA Training Cohort. Patients initially diagnosed with MM were excluded, a subset of patients without progression status were excluded, and patients with IgM immunofixation were excluded. Additionally, data from lab visits that occurred after the date of progression to MM were excluded. For patients that were treated before progression to MM, data from lab visits once they received treatment was censored. After applying these exclusions, the Training Cohort consisted of 1217 patients in total, with 172 progressing to MM.

### Comparison to Existing Models

The representativeness of the patient population was evaluated by applying current risk stratification models of SMM and MGUS to the PANGEA Training and Validation cohorts. Specifically, the 2018 Mayo Criteria 20/2/20 findings in SMM patients of the PANGEA Project was replicated and it was demonstrated that patients with zero 20/2/20 risk factors exhibited lower rates of progression than patients with one risk factor, followed by patients with two or more risk factors (FIGS. 6A-6D). Additionally, the 2014 International Myeloma Working Group (IMWG) criteria was applied for MGUS patients of the PANGEA Project and it was demonstrated that patients defined as high-risk progressed more rapidly to SMM than patients without high-risk features (FIGS. 7A and 7B).

### Further Description of the PANGEA Model (Bone Marrow (BM) and No BM)

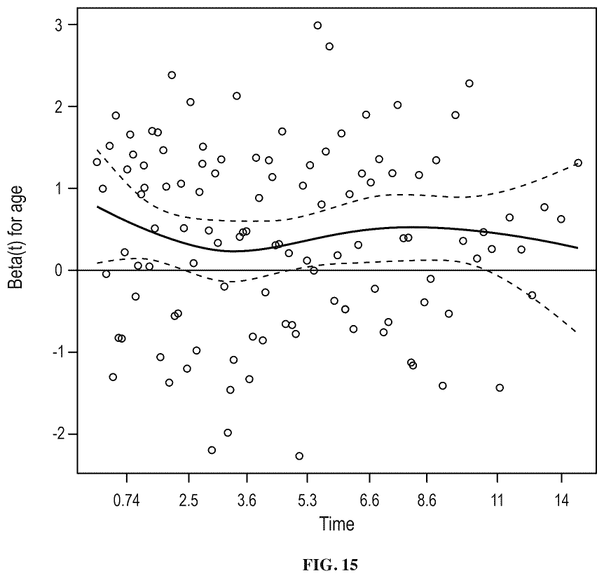

When evaluating models of progression risk on the Training Cohort, a set of categorical and continuous variables were evaluated. For categorical variables, the following were evaluated: history of hematological malignancy, race, sex, bisphosphonate treatment, immunofixation, and BMI. For continuous lab values, the following were evaluated: monoclonal protein, age, β2-microglobulin, creatinine, calcium, corrected calcium (total calcium+0.8*serum albumin), serum IgA, serum IgM, serum IgG, kappa free light chain (FLC), lambda FLC, FLC involve over uninvolved ratio, involved light chain, involved heavy chain, uninvolved light chain, LDH, albumin, and hemoglobin. Log 10 transformations were applied for the continuous variables of creatinine, calcium, corrected calcium, involved over uninvolved ratio, involved light chain, involved heavy chain, uninvolved light chain and LDH. Categorical trend variables captured at each clinical visit were also examined and current and previous measurements were used to determine if the biomarker presented similar measurements over time or if it was markedly increasing/decreasing. These categorical variables were defined through linear interpolation of the biomarkers' past measurements. After assessing the significance of the estimated biomarkers' trend (significance threshold set at 0.1), a categorical trend variable was included for decreasing hemoglobin. Additionally, production of the PANGEA Model (BM and No BM) was tested with and without imputation in the Training Cohort and found minimal differences in C-statistics between these Models (<2% change in C-statistics); ultimately, the Models were selected that did not use imputation. For each predictor it was also tested whether replacing the regression coefficient in the proportional hazard model with a more flexible function (i.e., f(x)) could capture potential non-linear relationships between predictor and progression to MM; an example of this is provided in FIG. 15. Importantly, none of the predictors used in the PANGEA Models improved prediction accuracy (increased C-statistic) in the Validation Cohorts when handled in this manner (Table 7).

To incorporate serial measurements and model trajectories of clinical biomarkers, regressions to individual lab values were linear fit at each visit date using data from a given visit and all prior visits. For each regression, the significance of the t-test for the slope was evaluated, and for regression results with p<0.1 and a positive slope, a trajectory indicator variable (trajij for variable i at timepoint j) was set to 1, and 0 otherwise. As this regression requires sufficient data to fit a linear regression, the trajectory variables were set to 0 for the first two timepoints for all variables for each patient. The trajectory variables were then computed at each time point for each lab value, and the trajectory variables were added as time-varying predictors to the Cox model.

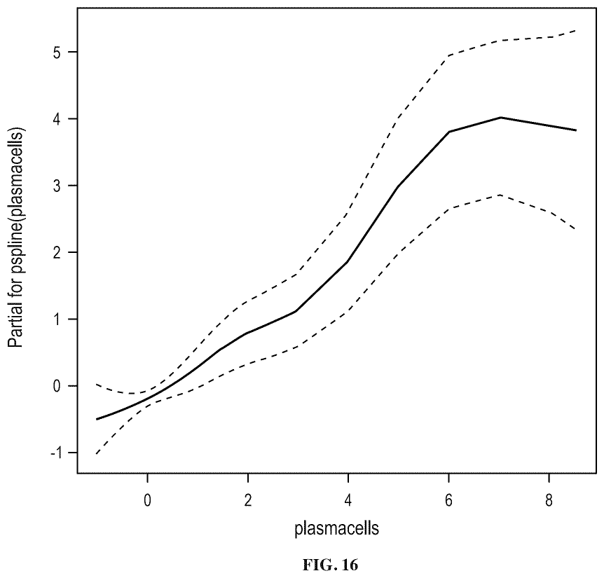

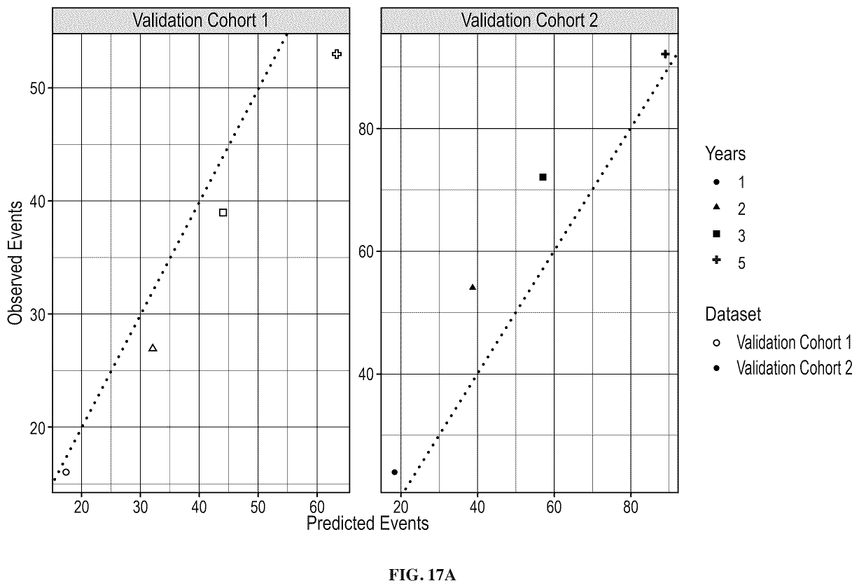

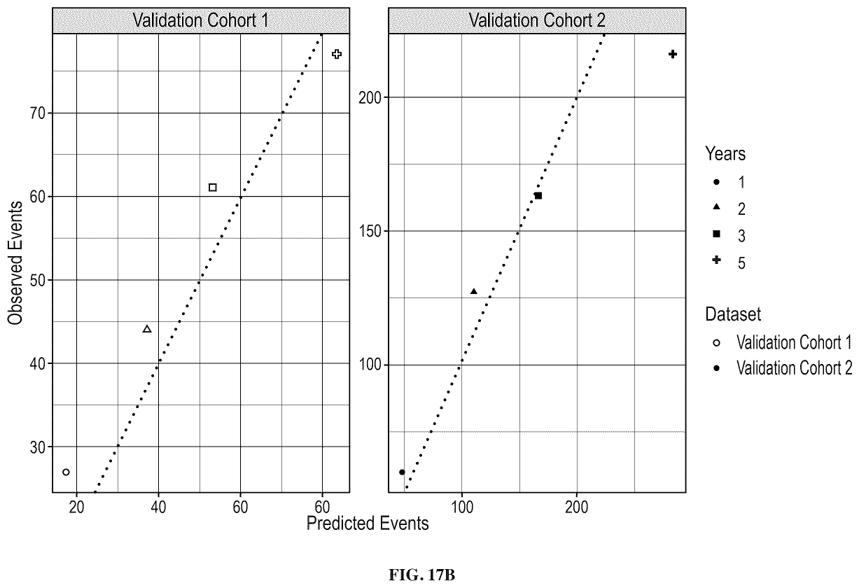

Finally, bootstrapping and calibration analyses were conducted for each PANGEA Model. To assess uncertainty on the concordance (C-statistics) between predictions in Validation Cohorts 1 and 2, a straightforward bootstrap procedure was used including two iterated steps: sampling with replacement the validation cohort (the size of the validation cohort and this resampled version are identical), and computations of C-statistics with splines for proportional hazards (see FIG. 16). Additionally, for Validation Cohorts 1 and 2, the calibration of the PANGEA Models was evaluated by computing the ratio between (i) number of predicted events (with event being progression from a precursor disease state to MM) between Visit 1 and Visit 2 and (ii) the number of actual events recorded (FIGS. 17A and 17B).

### Data Handling

Few biomarker inputs were missing in the Training and Validation datasets, and some of them were imputed by following common practices such as conducted for the 20/2/20 model (Lakshman A, et al., Blood Cancer J, 2018; 8:59.). The multivariable imputation by chained equations (MICE) package in R was used for all imputation processes with default settings. Specifically, if the BMPC % or the monoclonal protein were determined by the pathologist or SPEP, respectively, to be below the limit of detection (“Not Quantifiable”), then BMPC % was set to 1% (3% of all BMPC % in the Training Cohort) and M-spike to 0.01 g/dL (7% of all M-spikes in the Training Cohort) (the lower limits of these two tests). Similarly, BMPC % was set to 0% and M-spike to 0.00 g/dL if these variables were considered undetected in clinic. Overall, BMPC % was carried backwards for 90 days and forward for two years unless it was replaced by an antecedent or precedent measurement. For Validation Cohort 2, creatinine was missing at baseline for many patients, so this value was imputed using data on BMPC %, creatinine, age, monoclonal protein, and involved over uninvolved FLC ratio. The main outcome measure, time to progression, was defined as the time from precursor disease diagnosis per IMWG criteria to multiple myeloma diagnosis per SLIM-CRAB criteria.

### Statistical Analysis

To test sensitivity of the PANGEA Models, bootstrapping and calibration analyses (FIGS. 8A, 8B, 17A and 17B) and Schoenfeld tests, residual plots, and splines of predictors (Table 7 and FIGS. 15 and 16) were conducted. R version 4.2.0 was used for all statistical analysis. The average number of timepoints for Validation Cohort 1 is 6 and for Validation Cohort 2 is 1 (Table 5); thus, Validation Cohort 1 was used to validate how PANGEA perform for patients with long follow-up and Validation Cohort 2 to validate how PANGEA perform at diagnosis (Visit 1). When comparing to current risk stratification criteria, application of the IMWG (Kyle RA, et al., Leukemia, 2010; 24:1121-1127) or 20/2/20 (Lakshman A, et al., Blood Cancer J, 2018; 8:59) criteria as binary cutoffs at diagnosis was referred to as the Baseline Model and re-stratification by these criteria as discrete variables over time as the Rolling Model. Subcohorts of SMM patients from Validation Cohort 1 and Validation Cohort 2 were used for comparative analyses against the Baseline and Rolling 20/2/20 Models with or without bone marrow biopsies. This process was repeated for a subcohort of MGUS patients from Validation Cohort 2 for comparative analyses against the Baseline and Rolling IMWG Models.

TABLE 7
Schoenfeld tests to evaluate the assumption of constant effects of
the covariates on the risk of precursor disease progression to MM.

| Predictor | Chi Square | DF | p-value |
|---|---|---|---|
| FLC Ratio | 2.198 | 1 | 0.138 |
| (logged) | | | |
| M-spike | 1.456 | 1 | 0.227 |
| Age | 0.084 | 1 | 0.772 |
| Creatinine | 0.874 | 1 | 0.35 |
| (logged) | | | |
| BMPC% | 0.03 | 1 | 0.861 |
| Hemoglobin | 0.175 | 1 | 0.676 |
| Trajectory | | | |
| Global: | 5.752 | 6 | 0.452 |

A C-statistic is a standard metric used to compare prediction models: a C-statistic of 0·5 indicates that the model performs no better than random chance and a C-statistic of 1 indicates perfect prediction. For the PANGEA Models, C-statistics were computed for Visits 1, 2, and 3 for Validation Cohort 1 and at Visit 1 for Validation Cohort 2 (Table 2). For the Baseline Models, a Cox model was fit in the Training Cohort to estimate the hazard ratios for risk groups and the Cox linear predictor and C-statistics was computed in the Validation Cohorts. For the Rolling Models, a time-varying Cox model was fit in the Training Cohort to estimate hazard ratios and computed the linear predictor and C-statistic at Visit 1, 2, and 3 in Validation Cohort 1 (Table 2). The C-statistic estimates for Validation Cohort 1 and Validation Cohort 2 were representative of model accuracy in two cohorts independent from the Training Cohort used for developing the PANGEA Models.

To visualize the time to progression for the validation cohorts, patients were divided into quartiles (Low, Intermediate-Low, Intermediate-High and High) based on their predicted risk from the PANGEA Models. This discretization was only used when needed for graphical summaries and for comparisons with models that define risk groups. These groups were visualized using Kaplan-Meier curves for time to progression or death (with patients censored at treatment). In these analyses, patients were included who qualified for the PANGEA Models by having all necessary biomarker values available at the visit of interest (FIGS. 3A-3B).

It was explored if FISH biomarkers could provide additional prediction improvements to the PANGEA Model (BM). Due to the frequent absence and/or failure of FISH testing and rarity of some cytogenetic alterations, the training cohort was of limited size. Therefore, Training Cohort, Validation Cohort 1, and Validation Cohort 2 were combined for this analysis and patients with one or more successful FISH panels and corresponding laboratory datasets were selected, resulting in a subcohort of patients (Table 4). The PANGEA Model (FISH) was built as described herein, selecting for significant predictors (age, FLC ratio, M-spike in g/dL, creatinine in mg/dL, BMPC %, −17/17p, +1q, −13/13q, traj_{hemoglobin}).

### Further Description of the PANGEA Model (Fluorescence In Situ Hybridization (FISH))

For patients with both BMs and FISH panel results, all bone marrow biopsies after March 2020 had flow sorting performed with CD38, CD138, CD45, CD56, and CD319 probes. If ≥0.1% monotypic plasma cells were detected, FISH testing was performed as described in the PCPDS assay guidelines: mayocliniclabs.com/test-catalog/Overview/606079. Negative results were carried back and forward until replaced by a positive result, and positive results were carried forward. Positive results for primary events (+3/+7, +9/+15, trisomy 4, trisomy 12, trisomy 18, t(11;14), t(4;14), t(6;14), t(14;16), t(14;20), −13/13q deletion) were carried backward to baseline. Positive results for secondary events (−17/17p, +1q, and 8q24/MYC rearrangement) were not carried backward. All non-detected FISH findings were set as NAs and positive FISH results with total counts across the PANGEA Project <20 were considered not powered and excluded from analysis. To create the PANGEA Model (FISH), all available FISH findings were added as variables to the PANGEA Model (BM) through forward variable selection, and it was assessed whether additional variables were significant in this multivariate model (Wald test p<0.05).

### Further Description of the PANGEA App (the Pangea App)

An interactive website was created to evaluate patient risk using the PANGEA (BM) or PANGEA (No BM) model. Users could enter the model variables (monoclonal protein, involved over uninvolved kappa free light chain (FLC) ratio, creatinine, hemoglobin, and age). If BMbx data was available, users could enter this information and patient progression risk would be evaluated using the PANGEA (BM) model. Alternatively, if bone marrow biopsy (BMbx) data was not available, users could enter all other variables, and patient progression risk would be evaluated using the PANGEA (No BM) model. If longitudinal measurements were available, users could enter variables at multiple time points. If three or more time points were entered, the trajectories of hemoglobin, involved over uninvolved kappa free light chain (FLC) ratio, and creatinine would be evaluated using the regression procedure described above. The PANGEA Model allowed input of past and present patient measurements and output the probability of progression to MM at 1, 2, 5, 10, and 25 years regardless of whether the PANGEA (BM) or PANGEA (No BM) Model was used. If a variable was missing, a distribution of possible values for the missing variable based on the other variables was plotted, and users could enter different values for the missing variable to estimate the potential risk of progression. A model was computed for each possible missing marker, given the other variables, using the R package Bayesian Additive Regression Trees (BART). The user could then choose a value for the missing variable, and the probability of progression was evaluated using this value with either the PANGEA (BM) or PANGEA (No BM) depending on the availability of BMbx data.

### Other Embodiments

From the foregoing description, it will be apparent that variations and modifications may be made to the disclosure described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

All patents and publications mentioned in this specification are herein incorporated by reference to the same extent as if each independent patent and publication was specifically and individually indicated to be incorporated by reference.

What is claimed is:

1. A method for determining the risk that a patient with a multiple myeloma (MM) precursor disease will progress to MM, the method comprising:
a) analyzing the age of the patient and a plurality of values using at least one model trained to evaluate risk of an MM precursor disease progressing into MM, the plurality of values comprising:
a plurality of numeric values each being for a corresponding time-varying marker and at least one trajectory value describing a change over time of a time-varying marker;
b) generating, as a result of the analyzing using the at least one trained model, a numeric value indicating the risk that the MM precursor disease of the patient will progress into MM; and
c) outputting the value indicating the risk for the patient.

2. A method comprising:
assessing, with at least one processor and for a patient with a multiple myeloma (MM) precursor disease, a risk that the MM precursor disease of the patient will progress into MM, the assessing comprising:
analyzing, for the patient, an age of the patient together with a plurality of values each representing a level, detected at a time and for the patient, of a time-varying marker of a plurality of time-varying markers, the analyzing comprising analyzing the age of the patient and the plurality of values using at least one trained model trained to evaluate risk of MM precursor disease progressing into MM, the plurality of values comprising:
a first plurality of numeric values each being for a corresponding time-varying marker of a plurality of first time-varying markers, and
at least one trajectory value each describing a change over time of a corresponding time-varying marker of at least one second time-varying marker;
generating, as a result of the analyzing using the at least one trained model, a value indicating the risk that the MM precursor disease of the patient will progress into MM; and
outputting the value indicating the risk for the patient.

3. The method of claim 1, wherein the time-varying markers are clinical variables selected from creatinine, age, hemoglobin, M-spike, serum free light chain (FLC) ratio, bone marrow plasma cell percent (BMPC %), total protein, IgA, IgM, IgG, kappa free light chain (FLC), lambda FLC, calcium, albumin, hemoglobin, LDH, beta-2 microglobulin, and weight.

4. The method of claim 1, wherein the MM precursor disease is monoclonal gammopathy of undetermined significance (MGUS) or smoldering multiple myeloma (SMM).

5. The method of claim 2, wherein assessing the risk that the MM precursor disease of the patient will progress into MM comprises assessing the risk that the MM precursor disease of the patient will, within a timeframe, progress into MM.

6. The method of claim 5, wherein assessing the risk that the MM precursor disease of the patient will, within the timeframe, progress into MM, comprises assessing a risk for each of a plurality of timeframes that the MM precursor disease will, within the corresponding timeframe, progress into MM.

7. The method of claim 1, wherein:
generating the value indicating the risk comprises generating a numeric value indicating the risk; and
outputting the value indicating the risk comprises outputting the numeric value.

8. The method of claim 2, wherein:
assessing the risk further comprises determining whether an input value resulting from a bone marrow biopsy has been received; and
analyzing using the at least one trained model comprises:
in response to determining that an input value resulting from a bone marrow biopsy has been received, analyzing using a first trained model the age of the patient together with the plurality of values and the input value; and
in response to determining that an input value resulting from a bone marrow biopsy has not been received, analyzing the age of the patient together with the plurality of values using a second trained model different from the first trained model.

9. The method of claim 2, wherein analyzing the plurality of values each representing a level of a time-varying marker of the plurality of time-varying markers comprises analyzing a plurality of values each representing a detected level in a biological sample of a patient of a time-varying marker of the plurality of time-varying markers.

10. The method of claim 9, further comprising:
detecting the level of each of the plurality of time-varying markers in the biological sample of the patient; and
for a third time-varying marker of the at least one second time-varying marker, comparing levels detected over time in the biological sample of the patient of the third time-varying marker and determining the trajectory value describing the change over time of the third time-varying marker.

11. The method of claim 2, wherein:
the at least one second time-varying marker comprises a third time-varying marker; and
the trajectory value describing the change over time of the third time-varying marker indicates whether a value in the biological sample of the patient of the third time-varying marker has increased over time or decreased over time.

12. The method of claim 1, wherein analyzing, with the at least one trained model, the age of the patient together with the plurality of values each representing a level of a time-varying marker of the plurality of time-varying markers comprises analyzing, with the at least one trained model:
an age of the patient;
a free light chain (FLC) ratio for the patient;
a level of M-spike for the patient;
a level of creatinine for the patient; and
a trajectory value indicating whether an amount of hemoglobin increased or decreased.

13. The method of claim 2, wherein:
the first plurality of numeric values comprises a value that is a ratio of detected levels for the patient of two time-varying markers; and
analyzing the age together with the plurality of values using the at least one trained model comprises analyzing the ratio using the at least one trained model.

14. The method of claim 1, further comprising:
in response to determining that one of the plurality of values has not been received, determining a value to be used in the analyzing for the one of the plurality of values.

15. The method of claim 14, wherein:
a) determining the value to be used in the analyzing comprises determining the value based on at least one of the plurality of values that were received; or
b) determining the value to be used in the analyzing comprises determining the value to be a configured value.

16. The method of claim 2, wherein the analyzing, using the at least one trained model, the age and the plurality of values each representing the level of the time-varying marker comprises analyzing the age, the plurality of values, and at least one indicator of whether the patient has been detected to have at least one genetic marker.

17. The method of claim 16, wherein analyzing the age, the plurality of values, and the at least one indicator of whether the patient has been detected to have the at least one genetic marker comprises:
analyzing the age, the plurality of values, and at least one indicator each indicating whether the patient has been found to have a genetic marker selected from the group consisting of 17 deletion, 17p deletion, 13 deletion, 13q deletion, and 1q gain.

18. The method of claim 16, wherein analyzing the age, the plurality of values, and the at least one indicator of whether the patient has been detected to have the at least one genetic marker comprises:
analyzing the age, the plurality of values, and a plurality of indicators each indicating whether a patient has been found to have a corresponding one of 17 deletion, 17p deletion, 13 deletion, 13q deletion, and/or 1q gain.

19. A computer-implemented method for assessing risk for multiple myeloma precursor disease progression in a subject, the computer-implemented method comprising:
receiving, by at least one server from a computing device via a network, a multiple myeloma precursor disease progression request comprising: at least one variable representing at least one marker measurement associated with a subject;
determining, by the at least one server, a Precursor Asymptomatic Neoplasms by Group Effort Analysis (PANGEA) machine learning model from a set of PANGEA machine learning models based at least in part on the at least one variable provided in the multiple myeloma precursor disease progression request;
wherein each PANGEA machine learning model of the set of PANGEA machine learning models comprises trained PANGEA parameters trained for a particular combination of variables based at least in part on training data;
wherein the training data comprises historical marker measurements for the particular combination of variables paired with known trajectories of the particular combination of variables;
utilizing, by the at least one server, the PANGEA machine learning model to ingest the at least one variable and produce a predicted risk throughout a set of prediction periods representing a future risk of progression for the subject based at least in part on the trained PANGEA parameters; and
transmitting, by the at least one server, a multiple myeloma precursor disease progression response comprising the predicted risk throughout the prediction period,
the multiple myeloma precursor disease progression response being configured to cause the computing device to render a graphical plot depicting the future risk of progression based on the at least one continuous variable.

20. The computer-implemented method of claim 19, wherein:
a) the multiple myeloma precursor disease progression request comprises at least one variable selected from the group consisting of free light chain (FLC) ratio, M-spike level, age, creatinine level, and hemoglobin level;
b) the multiple myeloma precursor disease progression request comprises two or more of the following variables: free light chain (FLC) ratio, M-spike level, age, creatinine level, and hemoglobin level;
c) the multiple myeloma precursor disease progression request comprises three or more of the following variables: free light chain (FLC) ratio, M-spike level, age, creatinine level, and hemoglobin level;
d) the multiple myeloma precursor disease progression request comprises the following variables: free light chain (FLC) ratio, M-spike level, age, creatinine level, and hemoglobin level; or
e) the multiple myeloma precursor disease progression request further comprises the following variable: bone marrow plasma cell percent (BMPC %).
